(19) United States
(12) Patent Application Publication
(10) Pub. No.: US 2005/0070543 A1
(43) Pub. Date: Mar. 31, 2005

(54) Compositions of a chromene or phenyl acetic acid cyclooxygenase-2 selective inhibitor and an ACE inhibitor for the treatment of central nervous system damage

(75) Inventor: Diane T. Stephenson, Groton, CT (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/887,022

(22) Filed: Jul. 8, 2004

Related U.S. Application Data
(60) Provisional application No. 60/486,300, filed on Jul. 11, 2003.

(51) Int. Cl. A61K 31/50; A61K 31/415; A61K 31/366

(52) U.S. Cl. 514/247; 514/406; 514/457; 514/469

(57) ABSTRACT

The present invention provides compositions and methods for the treatment of central nervous system damage in a subject. More particularly, the invention provides a combination therapy for the treatment of a central nervous system ischemic condition or a central nervous system traumatic injury comprising the administration to a subject of an ACE inhibitor in combination with a chromene or phenyl acetic acid cyclooxygenase-2 selective inhibitor.

### CROSS REFERENCE TO RELATED APPLICATION

This application claims priority from Provisional Application Ser. No. 60/486,300, filed on Jul. 11, 2003, which is hereby incorporated by reference in its entirety.

### FIELD OF THE INVENTION

The present invention provides compositions and methods for the treatment of central nervous system damage. More particularly, the invention is directed toward a combination therapy for the treatment or prevention of ischemic-mediated central nervous system damage including ischemic stroke, or central nervous system damage resulting from traumatic injury, comprising the administration to a subject of an ACE inhibitor in combination with a chromene or phenyl acetic acid cyclooxygenase-2 selective inhibitor.

### BACKGROUND OF THE INVENTION

The continued increase in the incidence of ischemic-mediated central nervous system damage, including ischemic stroke, provides compelling evidence that there is a continuing need for better treatment strategies. Stroke, for example, is consistently the second or the third leading cause of death annually and the leading producer of disability among adults in the United States and western countries. Moreover, roughly 10% of patients with stroke become heavily handicapped, often needing attendant care.

Within the 1990's decade, the pathology underlying ischemic-mediated central nervous system injury was elucidated. Generally speaking, the normal amount of perfusion to brain gray matter is 60 to 70 mL/100 g of brain tissue/min. Death of central nervous system cells typically occurs only when the flow of blood falls below a certain level (approximately 8-10 mL/100 g of brain tissue/min) while at slightly higher levels the tissue remains alive but not able to function. For example, most strokes culminate in a core area of cell death (infarction) in which blood flow is so drastically reduced that the cells usually cannot recover. This threshold seems to occur when cerebral blood flow is 20 percent of normal or less. Without neuroprotective agents, nerve cells facing 80 to 100 percent ischemia will be irreversibly damaged within a few minutes. Surrounding the ischemic core is another area of tissue called the “ischemic penumbra” or “transitional zone” in which cerebral blood flow is between 20 and 50 percent of normal. Cells in this area are endangered, but not yet irreversibly damaged. Thus in the acute stroke, the affected central core brain tissue may die while the more peripheral tissues remain alive for many years after the initial insult, depending on the amount of blood the brain tissue receives.

At the cellular level, if left untreated, brain or spinal injury and death progress in a stepwise manner, rapidly within the core infarction, and over time within the ischemic penumbra. Without adequate blood supply, brain or spinal cells lose their ability to produce energy, particularly adenosine triphosphate (ATP). When this energy failure occurs, brain or spinal cells become damaged and will die if critical thresholds are reached. Immediate cell death within the ischemic core is typically necrotic, while cell death in the penumbra may be either necrotic or apoptotic. It is believed that there are an immense number of mechanisms at work causing brain or spinal cell damage and death following energy failure. Each of these mechanisms represents a potential route for intervention. One of the ways brain cells respond to energy failure is by elevating the concentration of intracellular calcium. Worsening this and driving the concentrations to dangerous levels is the process of excitotoxicity, in which brain cells release excessive amounts of glutamate, a neurotransmitter. This stimulates chemical and electrical activities in receptors on other brain cells, which leads to the degradation and destruction of vital cellular structures. Brain cells ultimately die as a result of the actions of calcium-activated proteases (enzymes which digest cell proteins), lipases (enzymes which digest cell membranes) and free radicals formed as a result of the ischemic cascade.

Interventions have been directed toward salvaging the ischemic penumbra and reducing its size. Restoration of blood flow is the first step toward rescuing the tissue within the penumbra. Therefore, timely recanalization of an occluded vessel to restore perfusion in both the penumbra and in the ischemic core is one treatment option employed. Partial recanalization also markedly reduces the size of the penumbra as well. Moreover, intravenous tissue plasminogen activator and other thrombolytic agents have been shown to have clinical benefit if they are administered within a few hours of symptom onset. Beyond this narrow time window, however, the likelihood of beneficial effects is reduced and hemorrhagic complications related to thrombolytic agents become excessive, seriously compromising their therapeutic value. Hypothermia decreases the size of the ischemic insult in both anecdotal clinical and laboratory reports. In addition, a wide variety of agents have been shown to reduce infarct volume in animal models. These agents include pharmacologic interventions that involve thrombolysis, calcium channel blockade, and cell membrane receptor antagonism. Successful treatment of stroke victims remains a high-unmet medical need. To date, no effective neuroprotective therapy exists to treat stroke. There is a continuing need for improved treatment regimes following ischemic-mediated central nervous system injury.

Neuroprotective agents have been shown to extend the time during which neurons within the ischemic penumbra remain viable (Albers, (1997) Am. J. Cardiol. 804(4C):4d-10d). Toward that end, several studies indicate that treatment with an ACE inhibitor following ischemic-mediated central nervous system injury may be beneficial. In particular, it has been suggested that ACE inhibitors have shown neuroprotective effect in animal models of ischemia. In one study, for example, it was demonstrated that treatment with an ACE inhibitor reduced the percentage of damaged neurons, as well as mitochondrial reactive oxygen species generation induced by glutamate or staurosporine and significantly reduced brain damage after focal ischemia as compared to control animals (Junker, et al., (1999) Eur. J. Pharmacol.;May 28(373) (1):21-33). Another study demonstrated a reduction in infarct volume to ischemic rats administered the ACE inhibitor trandolapril (Okamoto, et al., (2002) Hypertens. Res.;July 25(4):583-88). A further study demonstrated that ACE inhibitor administration improved endothelial function in a rat model of ischemia-reperfusion (Zhu, et al., (2003) J. Renin Angiotensin Aldosterone Syst.;March 4(1):31-7).

Since damage in the ischemic penumbra is associated with a heterogeneous cascade of molecular events, experts presently believe that treatment will not come by way of a single “magic bullet.” Instead, a combination of compounds that treat different components of the molecular cascade is likely to be the most effective method. (Zebrack, J. et al, (2002) Prog. Cardiovasc. Nurs 17(4):174-185). Several studies indicate that cyclooxygenase-2 is involved in the inflammatory component of the ischemic cascade. Cyclooxygenase-2 expression is known to be induced in the central nervous system following ischemic injury. In one study, it was shown that treatment with a cyclooxygenase-2 selective inhibitor reduced infarct volume in mice subjected to ischemic brain injury (Nagayama et al., (1999) J. Cereb. Blood Flow Metab.19(11):1213-19). A similar study showed that cyclooxygenase-2 deficient mice have a significant reduction in brain injury produced by occlusion of the middle cerebral artery when compared to mice that express cyclooxygenase-2 (ladecola et al., (2001) PNAS 98:1294-1299). Another study demonstrated that treatment with cyclooxygenase-2 selective inhibitor results in improved behavioral deficits induced by reversible spinal ischemia in rabbits (Lapchak et al., (2001) Stroke 32(5):1220-1230).

### SUMMARY OF THE INVENTION

Among the several aspects of the invention is provided a method and a composition for the treatment of reduced blood flow to the central nervous system in a subject. The composition comprises a chromene or phenyl acetic acid cyclooxygenase-2 selective inhibitor or an isomer, ester, a pharmaceutically acceptable salt or a prodrug thereof and an ACE inhibitor or an isomer, a pharmaceutically acceptable salt, ester, or prodrug thereof. The method comprises administering to the subject a chromene or phenyl acetic acid cyclooxygenase-2 selective inhibitor or an ismer, ester, a pharmaceutically acceptable salt or a prodrug thereof in combination with an ACE inhibitor or an isomer, a pharmaceutically acceptable salt, ester, or prodrug thereof.

In one embodiment, the cyclooxygenase-2 selective inhibitor or an isomer, ester, a pharmaceutically acceptable salt or a prodrug thereof is a member of the chromene class of compounds. For example, the chromene compound or an isomer, a pharmaceutically acceptable salt, ester, or prodrug thereof may be a compound of the formula: 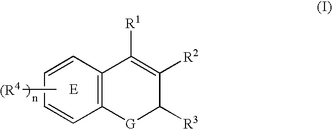
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or wherein R⁴ together with the carbon atoms to which it is attached and the remainder of ring E forms a naphthyl radical.

In another embodiment, the cyclooxygenase-2 selective inhibitor or an isomer, ester, a pharmaceutically acceptable salt or a prodrug thereof is a compound of the formula: 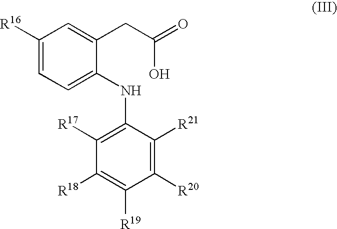
wherein
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl,
provided that R¹⁷, R¹⁸, R²⁰ and R²¹ are not all fluoro when R¹⁶ is ethyl and R¹⁹ is H.

In one embodiment, the ACE inhibitor is captopril.

In another embodiment, the ACE inhibitor is enalapril.

In another embodiment, the ACE inhibitor is benzapril.

Other aspects of the invention are described in more detail below.

### ABBREVIATIONS AND DEFINITIONS

The term “ACE inhibitor” also known as angiotensin converting enzyme inhibitor generally refers to compounds that are capable of inhibiting or disrupting the enzymatic conversion of angiotensin I to angiotensin II.

The term “acyl” is a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, and trifluoroacetyl.

The term “alkenyl” is a linear or branched radical having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl. The terms “alkenyl” and “lower alkenyl” also are radicals having “cis” and “trans” orientations, or alternatively, “E” and “Z” orientations. The term “cycloalkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The terms “alkoxy” and “alkyloxy” are linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” is an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “alkoxycarbonyl” is a radical containing an alkoxy radical, as defined above, attached via an oxygen atom to a carbonyl radical. More preferred are “lower alkoxycarbonyl” radicals with alkyl porions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

Where used, either alone or within other terms such as “haloalkyl”, “alkylsulfonyl”, “alkoxyalkyl” and “hydroxyalkyl”, the term “alkyl” is a linear, cyclic or branched radical having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkylamino” is an amino group that has been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “alkylaminoalkyl” is a radical having one or more alkyl radicals attached to an aminoalkyl radical.

The term “alkylaminocarbonyl” is an aminocarbonyl group that has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl”. radicals with lower alkyl portions as defined above.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “alkylthio” is a radical containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” is a radical containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” is a radical containing a linear or branched alkyl radical, of one to ten carbon atoms, attached to a divalent S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “alkynyl” is a linear or branched radical having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The term “aminoalkyl” is an alkyl radical substituted with one or more amino radicals. More preferred are “lower aminoalkyl” radicals. Examples of such radicals include aminomethyl, aminoethyl, and the like.

The term “aminocarbonyl” is an amide group of the formula —C(═O)NH₂.

The term “aralkoxy” is an aralkyl radical attached through an oxygen atom to other radicals.

The term “aralkoxyalkyl” is an aralkoxy radical attached through an oxygen atom to an alkyl radical.

The term “aralkyl” is an aryl-substituted alkyl radical such as benzyl, diphenylmethyl, triphenylmethyl, phenylethyl, and diphenylethyl. The aryl in said aralkyl may be additionally substituted with halo, alkyl, alkoxy, haloalkyl and haloalkoxy. The terms benzyl and phenylmethyl are interchangeable.

The term “aralkylamino” is an aralkyl radical attached through an amino nitrogen atom to other radicals. The terms “N-arylaminoalkyl” and “N-aryl-N-alkyl-aminoalkyl” are amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aralkylthio” is an aralkyl radical attached to a sulfur atom.

The term “aralkylthioalkyl” is an aralkylthio radical attached through a sulfur atom to an alkyl radical.

The term “aroyl” is an aryl radical with a carbonyl radical as defined above. Examples of aroyl include benzoyl, naphthoyl, and the like and the aryl in said aroyl may be additionally substituted.

The term “aryl”, alone or in combination, is a carbocyclic aromatic system containing one, two or three rings wherein such rings may be attached together in a pendent manner or may be fused. The term “aryl” includes aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “arylamino” is an amino group, which has been substituted with one or two aryl radicals, such as N-phenylamino. The “arylamino” radicals may be further substituted on the aryl ring portion of the radical.

The term “aryloxyalkyl” is a radical having an aryl radical attached to an alkyl radical through a divalent oxygen atom.

The term “arylthioalkyl” is a radical having an aryl radical attached to an alkyl radical through a divalent sulfur atom.

The term “carbonyl”, whether used alone or with other terms, such as “alkoxycarbonyl”, is —(C═O)—.

The terms “carboxy” or “carboxyl”, whether used alone or with other terms, such as “carboxyalkyl”, is —CO₂H.

The term “carboxyalkyl” is an alkyl radical substituted with a carboxy radical. More preferred are “lower carboxyalkyl” which are lower alkyl radicals as defined above, and may be additionally substituted on the alkyl radical with halo. Examples of such lower carboxyalkyl radicals include carboxymethyl, carboxyethyl and carboxypropyl.

The term “cycloalkenyl” is a partially unsaturated carbocyclic radical having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “cyclooxygenase-2 selective inhibitor” is a compound able to selectively inhibit cyclooxygenase-2 over cyclooxygenase-1. Typically, it includes compounds that have a cyclooxygenase-2 IC₅₀ of less than about 0.2 micro molar, and also have a selectivity ratio of cyclooxygenase-1 (COX-1) IC₅₀ to cyclooxygenase-2 (COX-2) IC₅₀ of at least about 5, more typically of at least about 50, and even more typically, of at least about 100. Moreover, the cyclooxygenase-2 selective inhibitors as described herein have a cyclooxygenase-1 IC₅₀ of greater than about 1 micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “halo” is a halogen such as fluorine, chlorine, bromine or iodine.

The term “haloalkyl” is a radical wherein any one or more of the alkyl carbon atoms is substituted with halo as defined above. Specifically included are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” is a radical having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “heteroaryl” is an unsaturated heterocyclyl radical. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1 H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4- thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also includes radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “heterocyclyl” is a saturated, partially unsaturated and unsaturated heteroatom-containing ring-shaped radical, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heterocyclylalkyl” is a saturated and partially unsaturated heterocyclyl-substituted alkyl radical, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, haloalkyl and haloalkoxy.

The term “hydrido” is a single hydrogen atom (H). This hydrido radical may be attached, for example, to an oxygen atom to form a hydroxyl radical or two hydrido radicals may be attached to a carbon atom to form a methylene (—CH2—) radical.

The term “hydroxyalkyl” is a linear or branched alkyl radical having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The term “pharmaceutically acceptable” is used adjectivally herein to mean that the modified noun is appropriate for use in a pharmaceutical product; that is the “pharmaceutically acceptable” material is relatively safe and/or non-toxic, though not necessarily providing a separable therapeutic benefit by itself. Pharmaceutically acceptable cations include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

The term “prodrug” refers to a chemical compound that can be converted into a therapeutic compound by metabolic or simple chemical processes within the body of the subject. For example, a class of prodrugs of COX-2 inhibitors is described in U.S. Pat. No. 5,932,598, herein incorporated by reference.

The term “subject” for purposes of treatment includes any human or animal who has reduced blood flow to the central nervous system. The subject can be a domestic livestock species, a laboratory animal species, a zoo animal or a companion animal. In one embodiment, the subject is a mammal. In another embodiment, the mammal is a human being.

The term “sulfonyl”, whether used alone or linked to other terms such as alkylsulfonyl, is a divalent radical —SO₂—. “Alkylsulfonyl” is an alkyl radical attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” are NH₂O₂S—.

The phrase “therapeutically-effective” is intended to qualify the amount of each agent (i.e. the amount of chromene or phenyl acetic acid cyclooxygenase-2 selective inhibitor and the amount of ACE inhibitor) which will achieve the goal of improvement in disorder severity and the frequency of incidence over no treatment or treatment of each agent by itself.

The term “treat” or “treatment” as used herein, includes administration of the combination therapy to a subject known to have a central nervous system disorder. In other aspects, it also includes either preventing the onset of a clinically evident central nervous system disorder altogether or preventing the onset of a preclinically evident stage of a central nervous system disorder subject. This definition includes prophylactic treatment.

The term “thrombotic event” or “thromboembolic event” includes, but is not limited to arterial thrombosis, including stent and graft thrombosis, cardiac thrombosis, coronary thrombosis, heart valve thrombosis, pulmonary thrombosis and venous thrombosis. Cardiac thrombosis is thrombosis in the heart. Pulmonary thrombosis is thrombosis in the lung. Arterial thrombosis is thrombosis in an artery. Coronary thrombosis is the development of an obstructive thrombus in a coronary artery, often causing sudden death or a myocardial infarction. Venous thrombosis is thrombosis in a vein. Heart valve thrombosis is a thrombosis on a heart valve. Stent thrombosis is thrombosis resulting from and/or located in the vicinity of a vascular stent. Graft thrombosis is thrombosis resulting from and/or located in the vicinity of an implanted graft, particularly a vascular graft. A thrombotic event as used herein is meant to embrace both a local thrombotic event and a distal thrombotic event occurring anywhere within the body (e.g., a thromboembolic event such as for example an embolic stroke).

The term “vaso-occlusive event” includes a partial occlusion (including a narrowing) or complete occlusion of a blood vessel, a stent or a vascular graft. A vaso-occlusive event intends to embrace thrombotic or thromboembolic events, and the vascular occlusion disorders or conditions to which they give rise. Thus, a vaso-occlusive event is intended to embrace all vascular occlusive disorders resulting in partial or total vessel occlusion from thrombotic or thromboembolic events.

### DESCRIPTION OF THE PREFERRED EMBODIMENTS

The present invention provides a combination therapy comprising the administration to a subject of a therapeutically effective amount of a chromene or phenyl acetic acid COX-2 selective inhibitor or an isomer, ester, a pharmaceutically acceptable salt or a prodrug thereof in combination with a therapeutically effective amount of an ACE inhibitor or an isomer, a pharmaceutically acceptable salt, ester, or prodrug thereof. The combination therapy may be used to treat or prevent damage to a central nervous system cell resulting from a reduction in blood flow or traumatic injury. When administered as part of a combination therapy, the chromene or phenyl acetic acid COX-2 selective inhibitor together with the ACE inhibitor provide enhanced treatment options as compared to administration of either the ACE inhibitor or the chromene or phenyl acetic acid COX-2 selective inhibitor alone.

### Chrome or Phenyl Acetic Acid Cyclooxygenase-2 Selective Inhibitors

A number of suitable chromene or phenyl acetic acid cyclooxygenase-2 selective inhibitors or pharmaceutically acceptable salts or prodrugs thereof may be employed in the composition of the current invention. In one embodiment the chromene or phenyl acetic acid cyclooxygenase-2 selective inhibitor is a chromene compound that is a substituted benzopyran or a substituted benzopyran analog, and even more typically, selected from the group consisting of substituted benzothiopyrans, dihydroquinolines, dihydronaphthalenes or a compound having Formula/shown below and possessing, by way of example and not limitation, the structures disclosed in Table 1x. Furthermore, benzopyran cyclooxygenase-2 selective inhibitors useful in the practice of the present methods are described in U.S. Pat. Nos. 6,034,256 and 6,077,850 herein incorporated by reference in their entirety.

In one embodiment, the chromene or phenyl acetic acid cyclooxygenase-2 selective inhibitor or a pharmaceutically acceptable salt or a prodrug thereof is a chromene compound represented by Formula I: 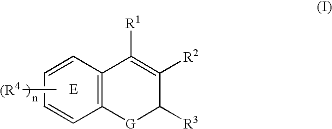
wherein n is an integer which is 0, 1, 2, 3 or 4;
wherein G is O, S or NRa;
wherein R^{a} is alkyl;
wherein R¹ is selected from the group consisting of H and aryl;
wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from the group consisting of alkylthio, nitro and alkylsulfonyl; and

wherein each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or wherein R⁴ together with the carbon atoms to which it is attached and the remainder of ring E forms a naphthyl radical.

The cyclooxygenase-2 selective inhibitor of the present invention may also be a compound of Formula (I) or an isomer, ester, a pharmaceutically acceptable salt, or a prodrug thereof wherein:
n is an integer which is 0, 1, 2, 3 or 4;
G is O, S or NR^{a};
R^{a} is alkyl;
R¹ is H;
R² is selected from the group consisting of carboxyl, lower alkyl, lower aralkyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from the group consisting of alkylthio, nitro and alkylsulfonyl; and
each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

In a further embodiment, the chromene or phenyl acetic acid cyclooxygenase-2 selective inhibitor may also be a compound of Formula (I), or an isomer, ester, a pharmaceutically acceptable salt, or a prodrug thereof; wherein:
n is an integer which is 0, 1, 2, 3 or 4;
G is oxygen or sulfur;
R¹ is H;
R² is carboxyl, lower alkyl, lower aralkyl or lower alkoxycarbonyl;
R³ is lower haloalkyl, lower cycloalkyl or phenyl; and
each R⁴ is independently H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or wherein R⁴ together with the carbon atoms to which it is attached and the remainder of ring E forms a naphthyl radical.

The chromene or phenyl acetic acid cyclooxygenase-2 selective inhibitor may also be a compound of Formula (I) or an isomer, ester, a pharmaceutically acceptable salt, or a prodrug thereof; wherein:
n is an integer which is 0, 1, 2, 3 or 4;
G is oxygen or sulfur;
R¹ is H;
R² is carboxyl;
R³ is lower haloalkyl; and
each R⁴ is independently H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The chromene or phenyl acetic acid cyclooxygenase-2 selective inhibitor may also be a compound of Formula (I) or an isomer, ester, a pharmaceutically acceptable salt or a prodrug thereof; wherein:
n is an integer which is 0, 1, 2, 3 or 4;
G is oxygen or sulfur;
R¹ is H;
R² is carboxyl;
R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and
each R⁴ is independently H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The chromene or phenyl acetic acid cyclooxygenase-2 selective inhibitor may also be a compound of Formula (I) or an isomer, ester, a pharmaceutically acceptable salt or a prodrug thereof; wherein:
n is an integer which is 0, 1, 2, 3 or4;
G is oxygen or sulfur;
R¹ is H;
R² is carboxyl;
R³ is trifluoromethyl or pentafluoroethyl; and
each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

In yet another embodiment, the chromene or phenyl acetic acid cyclooxygenase-2 selective inhibitor used in connection with the method(s) of the present invention can also be a compound having the structure of Formula (I) or an isomer, ester, a pharmaceutically acceptable salt or a prodrug thereof:
wherein:
n is 4;
G is O or S;
R¹ is H;
R² is CO₂H;
R³ is lower haloalkyl;
a first R⁴ corresponding to R⁹ is hydrido or halo;
a second R⁴corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;
a third R⁴ corresponding to R¹¹ is H, lower alkyl, halo, lower alkoxy, or aryl; and
a fourth R⁴ corresponding to R¹² is H, halo, lower alkyl, lower alkoxy, and aryl;
wherein Formula (I) is represented by Formula (Ia):

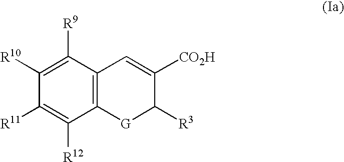

The chromene or phenyl acetic acid cyclooxygenase-2 selective inhibitor used in connection with the method(s) of the present invention can also be a compound of having the structure of Formula (Ia) or a pharmaceutically acceptable salt or a prodrug thereof; wherein:
R³ is trifluoromethyl or pentafluoroethyl;
R⁹ is H, chloro, or fluoro;
R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;
R¹¹ is H, methyl, ethyl, isopropyl, tert-butyl, chloro, methoxy, diethylamino, or phenyl; and
R¹² is H, chloro, bromo, fluoro, methyl, ethyl, tert-butyl, methoxy, or phenyl.

Examples of exemplary chromene cyclooxygenase-2 selective inhibitors are depicted in Table 1x below.

TABLE 1X
EXAMPLES OF CHROMENE CYCLOOXYGENASE-2 SELECTIVE
INHIBITORS AS EMBODIMENTS

| Compound | |
|---|---|
| Number | Structural Formula |
| B-3 | 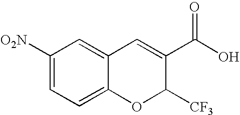 |
| | 6-Nitro-2-trifluoromethyl-2H-1- |
| | benzopyran-3-carboxylic acid |
| B-4 | 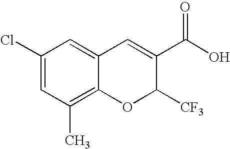 |
| | 6-Chloro-8-methyl-2-trifluoromethyl- |
| | 2H-1-benzopyran-3-carboxylic acid |
| B-5 | 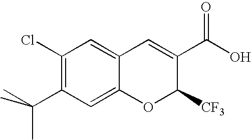 |
| | ((S)-6-Chloro-7-(1,1-dimethylethyl)-2- |
| | (trifluoromethyl-2H-1-benzopyran-3-carboxylic acid |
| B-6 | 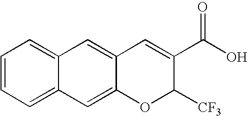 |
| | 2-Trifluoromethyl-2H-naphtho[2,3-b] |
| | pyran-3-carboxylic acid |
| B-7 | 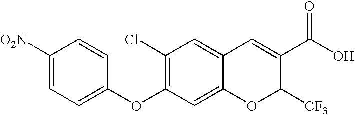 |
| | 6-Chloro-7-(4-nitrophenoxy)-2-(trifluoromethyl)-2H-1- |
| | benzopyran-3-carboxylic acid |
| B-8 | 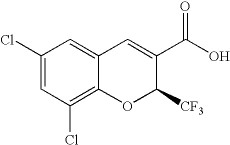 |
| | ((S)-6,8-Dichloro-2-(trifluoromethyl)- |
| | 2H-1-benzopyran-3-carboxylic acid |
| B-9 | 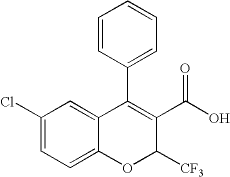 |
| | 6-Chloro-2-(trifluoromethyl)-4-phenyl-2H- |
| | 1-benzopyran-3-carboxylic acid |
| B-10 | 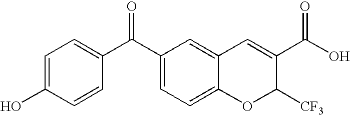 |
| | 6-(4-Hydroxybenzoyl)-2-(trifluoromethyl)- |
| | 2H-1-benzopyran-3-carboxylic acid |
| B-11 | 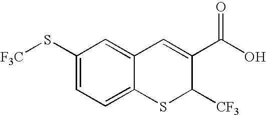 |
| | 2-(Trifluoromethyl)-6-[(trifluoromethyl)thiol- |
| | 2H-1-benzothiopyran-3-carboxylic acid |
| B-12 | 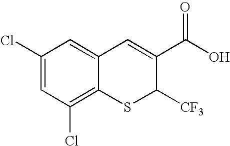 |
| | 6,8-Dichloro-2-trifluoromethyl-2H-1- |
| | benzothiopyran-3-carboxylic acid |
| B-13 | 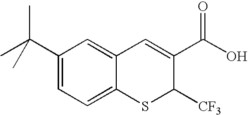 |
| | 6-(1,1-Dimethylethyl)-2-(trifluoromethyl)- |
| | 2H-1-benzothiopyran-3-carboxylic acid |
| B-14 | 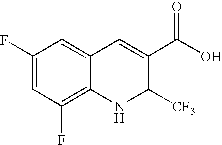 |
| | 6,7-Difluoro-1,2-dihydro-2-(trifluoromethyl)- |
| | 3-quinolinecarboxylic acid |
| B-15 | 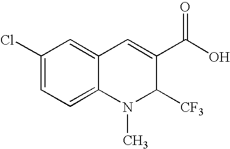 |
| | 6-Chloro-1,2-dihydro-1-methyl-2-(trifluoromethyl)- |
| | 3-quinolinecarboxylic acid |
| B-16 | 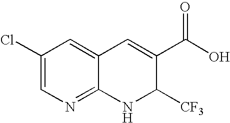 |
| | 6-Chloro-2-(trifluoromethyl)-1,2-dihydro |
| | [1,8]naphthyridine-3-carboxylic acid |
| B-17 | 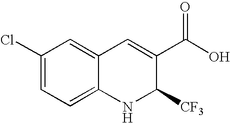 |
| | ((S)-6-Chloro-1,2-dihydro-2-(trifluoromethyl)- |
| | 3-quinolinecarboxylic acid |

In a further embodiment, the chromene or phenyl acetic acid cyclooxygenase-2 selective inhibitor or an isomer, ester, a pharmaceutically acceptable salt or a prodrug thereof used in connection with the method(s) of the present invention can be selected from the class of phenylacetic acid derivative cyclooxygenase-2 selective inhibitors represented by the general structure of Formula (III): 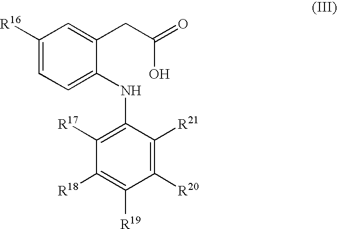
wherein
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl,
provided that R¹⁷, R¹⁸, R²⁰ and R²¹ are not all fluoro when R¹⁶ is ethyl and R¹⁹ is H.

Another phenylacetic acid derivative cyclooxygenase-2 selective inhibitor used in connection with the method(s) of the present invention is a compound that has the designation of COX 189 (B-211) and that has the structure shown in Formula (III) or an isomer, ester, a pharmaceutically acceptable salt or a prodrug thereof, wherein:
R¹⁶ is ethyl;
R¹⁷ and R¹⁹ are chloro;
R¹⁸ and R²⁰ are hydrogen; and
and R²¹ is methyl.

In yet another embodiment, the chromene or phenyl acetic acid cyclooxygenase-2 selective inhibitor or an isomer, ester, a pharmaceutically acceptable salt or a prodrug thereof is represented by Formula (IV): 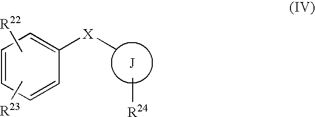
wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

According to another embodiment, the chromene or phenyl acetic acid cyclooxygenase-2 selective inhibitors or isomers, esters, pharmaceutically acceptable salts or prodrugs thereof used in the present method(s) have the structural Formula (V): 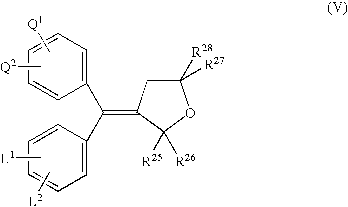
wherein:
T and M are independently phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or
R²⁵ and R²⁶, together with the carbon atom to which they are attached, form a carbonyl or a saturated hydrocarbon ring having from 3 to 7 carbon atoms; or
R²⁷and R²⁸, together with the carbon atom to which they are attached, form a carbonyl or a saturated hydrocarbon ring having from 3 to 7 carbon atoms;
Q¹, Q², L¹ or L² are independently hydrogen, halogen, lower alkyl having from 1 to 6 carbon atoms, trifluoromethyl, lower methoxy having from 1 to 6 carbon atoms, alkylsulfinyl or alkylsulfonyl; and
at least one of Q¹, Q², L¹ or L² is in the para position and is
—S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or Q¹ and Q² together form methylenedioxy; or L¹ and L² together form methylenedioxy.

In a further embodiment, compounds that are useful for the chromene or phenyl acetic acid cyclooxygenase-2 selective inhibitor or an isomer, ester, a pharmaceutically acceptable salt or a prodrug thereof in connection with the method(s) of the present invention, the structures for which are set forth in Table 3x below, include, but are not limited to:
6-chloro-2-trifluoromethyl-2H-1-benzopyran-3-carboxylic acid (B-27);
6-chloro-7-methyl-2-trifluoromethyl-2H-1-benzopyran-3-carboxylic acid (B-28);
8-(1 -methylethyl )-2-trifluoromethyl-2H-1-benzopyran-3-carboxylic acid (B-29);
6-chloro-8-(1-methylethyl)-2-trifluoromethyl-2H-1-benzopyran-3-carboxylic acid (B-30);
2-trifluoromethyl-3H-naphtho[2,1-b]pyran-3-carboxylic acid (B-31);
7-(1,1-dimethylethyl)-2-trifluoromethyl-2H-1-benzopyran-3-carboxylic acid (B-32);
6-bromo-2-trifluoromethyl-2H-1-benzopyran-3-carboxylic acid (B-33);
8-chloro-2-trifluoromethyl-2H-1-benzopyran-3-carboxylic acid (B-34);
6-trifluoromethoxy-2-trifluoromethyl-2H-1-benzopyran-3-carboxylic acid (B-35);
5,7-dichloro-2-trifluoromethyl-2H-1-benzopyran-3-carboxylic acid (B-36);
8-phenyl-2-trifluoromethyl-2H-1-benzopyran-3-carboxylic acid (B-37);
7,8-dimethyl-2-trifluoromethyl-2H-1-benzopyran-3-carboxylic acid (B-38);
6,8-bis(dimethylethyl)-2-trifluoromethyl-2H-1-benzopyran-3-carboxylic acid (B-39);
7-(1-methylethyl)-2-trifluoromethyl-2H-1-benzopyran-3-carboxylic acid (B40);
7-phenyl-2-trifluoromethyl-2H-1-benzopyran-3-carboxylic acid (B41);
6-chloro-7-ethyl-2-trifluoromethyl-2H-1-benzopyran-3-carboxylic acid (B42);
6-chloro-8-ethyl-2-trifluoromethyl-2H-1-benzopyran-3-carboxylic acid (B43);
6-chloro-7-phenyl-2-trifluoromethyl-2H-1-benzopyran-3-carboxylic acid (B44);
6,7-dichloro-2-trifluoromethyl-2H-1-benzopyran-3-carboxylic acid (B-45);
6,8-dichloro-2-trifluoromethyl-2H-1-benzopyran-3-carboxylic acid (B46);
6-chloro-8-methyl-2-trifluoromethyl-2H-1-benzopyran-3-carboxylic acid (B47);
8-chloro-6-methyl-2-trifluoromethyl-2H-1-benzopyran-3-carboxylic acid (B48)
8-chloro-6-methoxy-2-trifluoromethyl-2H-1-benzopyran-3-carboxylic acid (B49);
6-bromo-8-chloro-2-trifluoromethyl-2H-1-benzopyran-3-carboxylic acid (B-50);
8-bromo-6-fluoro-2-trifluoromethyl-2H-1-benzopyran-3-carboxylic acid (B-51);
8-bromo-6-methyl-2-trifluoromethyl-2H-1-benzopyran-3-carboxylic acid (B-52);
8-bromo-5-fluoro-2-trifluoromethyl-2H-1-benzopyran-3-carboxylic acid (B-53);
6-chloro-8-fluoro-2-trifluoromethyl-2H-1-benzopyran-3-carboxylic acid (B-54);
6-bromo-8-methoxy-2-trifluoromethyl-2H-1-benzopyran-3-carboxylic acid (B-55);
6-[[(phenylmethyl)amino]sulfonyl]-2-trifluoromethyl-2H-1-benzopyran-3-carboxylic acid (B-56);
6-[(dimethylamino)sulfonyl]-2-trifluoromethyl-2H-1-benzopyran-3-carboxylic acid (B-57);
6-[(methylamino)sulfonyl]-2-trifluoromethyl-2H-1-benzopyran-3-carboxylic acid (B-58);
6-[(4-morpholino)sulfonyl]-2-trifluoromethyl-2H-1-benzopyran-3-carboxylic acid (B-59);
6-[(1,1-dimethylethyl)aminosulfonyl]-2-trifluoromethyl-2H-1-benzopyran-3-carboxylic acid (B-60);
6-[(2-methylpropyl)aminosulfonyl]-2-trifluoromethyl-2H-1-benzopyran-3-carboxylic acid (B-61);
6-methylsulfonyl-2-trifluoromethyl-2H-1-benzopyran-3-carboxylic acid (B-62);
8-chloro-6-[[(phenylmethyl)amino]sulfonyl]-2-trifluoromethyl-2H-1-benzopyran-3-carboxylic acid (B-63);
6-phenylacetyl-2-trifluoromethyl-2H-1-benzopyran-3-carboxylic acid (B-64);
6,8-dibromo-2-trifluoromethyl-2H-1-benzopyran-3-carboxylic acid (B-65);
8-chloro-5,6-dimethyl-2-trifluoromethyl-2H-1-benzopyran-3-carboxylic acid (B-66);
6,8-dichloro-(S)-2-trifluoromethyl-2H-1-benzopyran-3-carboxylic acid (B-67);
6-benzylsulfonyl-2-trifluoromethyl-2H-1-benzopyran-3-carboxylic acid (B-68);
6-[[N-(2-furylmethyl)amino]sulfonyl]-2-trifluoromethyl-2H-1-benzopyran-3-carboxylic acid (B-69);
6-[[N-(2-phenylethyl)amino]sulfonyl]-2-trifluoromethyl-2H-1-benzopyran-3-carboxylic acid (B-70);
6-iodo-2-trifluoromethyl-2H-1-benzopyran-3-carboxylic acid (B-71);
7-(1,1-dimethylethyl)-2-pentafluoroethyl-2H-1-benzopyran-3-carboxylic acid (B-72);
6-chloro-2-trifluoromethyl-2H-1-benzothiopyran-3-carboxylic acid (B-73);
3-[(3-Chloro-phenyl)-(4-methanesulfonyl-phenyl)-methylene]-dihydro-furan-2-one or BMS-347070 (B-74);
8-acetyl-3-(4-fluorophenyl)-2-(4-methylsulfonyl)phenyl-imidazo(1,2-a)pyridine (B-75);
5,5-dimethyl-4-(4-methylsulfonyl)phenyl-3-phenyl-2-(5H)-furanone (B-76);
2-[1-[4-(methylsulfonyl)phenyl-4-(trifluoromethyl)-1H-imidazol-2-yl]pyridine (B-131);
2-methyl-4-[1-[4-(methylsulfonyl)phenyl-4-(trifluoromethyl)-1H-imidazol-2-yl]pyridine (B-132);
2-methyl-6-[1-[4-(methylsulfonyl)phenyl-4-(trifluoromethyl)-1H-imidazol-2-yl]pyridine (B-133);
2-(3-fluoro-4-methoxyphenyl)-1-[4-(methylsulfonyl)phenyl-4-(trifluoromethyl)-1H-imidazole (B-140);
6-chloro-7-(1,1-dimethylethyl)-2-trifluoromethyl-2H-1-benzopyran-3-carboxylic acid (B-196);
6-chloro-8-methyl-2-trifluoromethyl-2H-1-benzopyran-3-carboxylic acid (B-197);
5,5-dimethyl-3-(3-fluorophenyl)-4-methylsulfonyl-2(5H )-furanone (B-198);
6-chloro-2-trifluoromethyl-2H-1-benzothiopyran-3-carboxylic acid (B-199);
[2-(2-chloro-6-fluoro-phenylamino)-5-methyl-phenyl]-acetic acid or COX 189 (B-211);
N-(4-Nitro-2-phenoxy-phenyl)-methanesulfonamide or nimesulide (B-212);
N-[6-(2,4-difluoro-phenoxy)-1-oxo-indan-5-yl]-methanesulfonamide or flosulide (B-213);
N-[6-(2,4-Difluoro-phenylsulfanyl)-1-oxo-1H-inden-5-yl]-methanesulfonamide, sodium salt or L-745337 (B-214);
N-[5-(4-fluoro-phenylsulfanyl)-thiophen-2-yl]-methanesulfonamide or RWJ-63556 (B-215);
(5Z)-2-amino-5-[[3,5-bis(1,1-dimethylethyl)-4-hydroxyphenyl]methylene]-4(5H)-thiazolone or darbufelone (B-217);
CS-502 (B-218);
LAS-34475 (B-219);
LAS-34555 (B-220);
S-33516 (B-221);
SD-8381 (B-222);
L-783003 (B-223);
N-[3-(formylamino)-4-oxo-6-phenoxy4H-1-benzopyran-7-yl]-methanesulfonamide or T-614 (B-224);
D-1367 (B-225);
L-748731 (B-226);
(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);
CGP-28238 (B-228);

4-[[3,5-bis(1,1-dimethylethyl)-4-hydroxyphenyl]methylene]dihydro-2-methyl-2H-1,2-oxazin-3(4H)-one or BF-389 (B-229);
GR-253035 (B-230);
6-dioxo-9H-purin-8-yl-cinnamic acid (B-231);
S-2474 (B-232);
4-[4-(methyl)-sulfonyl)phenyl]-3-phenyl-2(5H)-furanone;
4-(5-methyl-3-phenyl4-isoxazolyl);
2-(6-methylpyrid-3-yl)-3-(4-methylsulfonylphenyl)-5-chloropyridine;
4-[5-(4-methylphenyl)-3-(trifluoromethyl)-1H-pyrazol-1-yl];
N-[[4-(5-methyl-3-phenyl-4-isoxazolyl)phenyl]sulfonyl];
(S)-6,8-dichloro-2-(trifluoromethyl)-2H-1-benzopyran-3-carboxylic acid;
2-(3,4-difluorophenyl)-4-(3-hydroxy-3-methylbutoxy)-5-[4-(methylsulfonyl)phenyl]-3(2H)-pyridzainone;
2-trifluoromethyl-3H-naptho[2,1-b]pyran-3-carboxylic acid;
6-chloro-7-(1,1-dimethylethyl)-2-trifluoromethyl-2H-1-benzopyran-3-carboxylic acid;

[2-(2,4-dichloro-6-ethyl-3,5-dimethyl-phenylamino)-5-propyl-phenyl]-acetic acid.

TABLE 3X
EXAMPLES OF CHROMENE OR PHENYL ACETIC ACID

CYCLOOXYGENASE-2 SELECTIVE INHIBITORS AS EMBODIMENTS

| Compound Number | Structural Formula |
|---|---|
| B-27 | 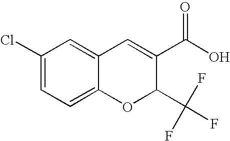 |
| | 6-chloro-2-trifluoromethyl-2H-1-benzopyran-3-carboxylic acid; |
| B-28 | 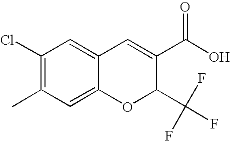 |
| | 6-chloro-7-methyl-2-trifluoromethyl-2H-1-benzopyran-3- |
| | carboxylic acid; |
| B-29 | 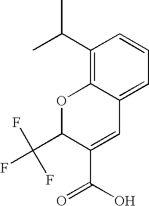 |
| | 8-(1-methylethyl)-2-trifluoromethyl-2H-1-benzopyran-3- |
| | carboxylic acid; |
| B-30 | 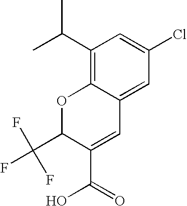 |
| | 6-chloro-8-(1-methylethyl)-2-trifluoromethyl- |
| | 2H-1-benzopyran-3-carboxylic acid; |
| B-31 | 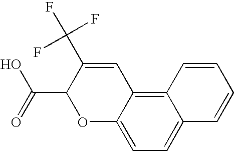 |
| | 2-trifluoromethyl-3H-naphtho[2,1-b]pyran-3-carboxylic acid; |
| B-32 | 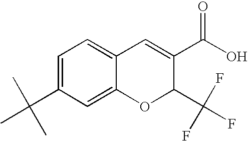 |
| | 7-(1,1-dimethylethyl)-2-trifluoromethyl-2H-1-benzopyran-3- |
| | carboxylic acid; |
| B-33 | 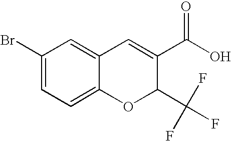 |
| | 6-bromo-2-trifluoromethyl-2H-1-benzopyran-3-carboxylic acid; |
| B-34 | 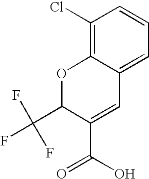 |
| | 8-chloro-2-trifluoromethyl-2H-1-benzopyran-3-carboxylic acid; |
| B-35 | 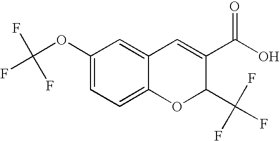 |
| | 6-trifluoromethoxy-2-trifluoromethyl-2H-1-benzopyran-3- |
| | carboxylic acid; |
| B-36 | 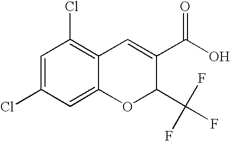 |
| | 5,7-dichloro-2-trifluoromethyl-2H-1-benzopyran-3-carboxylic acid; |
| B-37 | 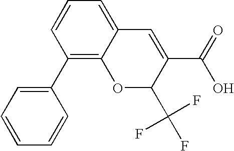 |
| | 8-phenyl-2-trifluoromethyl-2H-1-benzopyran-3-carboxylic acid; |
| B-38 | 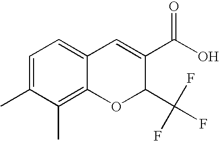 |
| | 7,8-dimethyl-2-trifluoromethyl-2H-1-benzopyran-3- |
| | carboxylic acid; |
| B-39 | 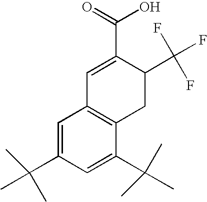 |
| | 6,8-bis(dimethylethyl)-2-trifluoromethyl-2H-1-benzopyran-3- |
| | carboxylic acid; |
| B-40 | 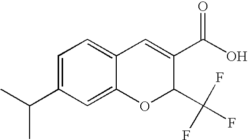 |
| | 7-(1-methylethyl)-2-trifluoromethyl-2H-1-benzopyran-3- |
| | carboxylic acid; |
| B-41 | 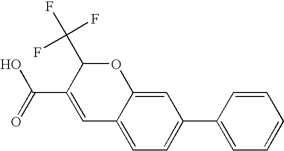 |
| | 7-phenyl-2-trifluoromcthyl-2H-1-benzopyran-3-carboxylic acid; |
| B-42 | 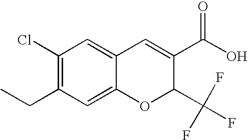 |
| | 6-chloro-7-ethyl-2-trifluoromethyl-2H-1-benzopyran-3- |
| | carboxylic acid; |
| B-43 | 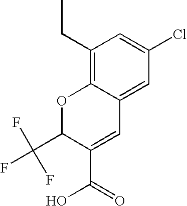 |
| | 6-chloro-8-ethyl-2-trifluoromethyl-2H-1-benzopyran-3- |
| | carboxylic acid; |
| B-44 | 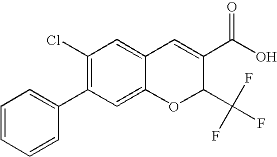 |
| | 6-chloro-7-phenyl-2-trifluoromethyl-2H-1-benzopyran-3- |
| | carboxylic acid; |
| B-45 | 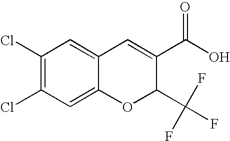 |
| | 6,7-dichloro-2-trifluoromethyl-2H-1-benzopyran-3-carboxylic acid; |
| B-46 | 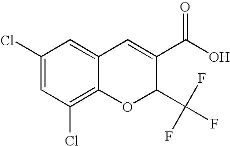 |
| | 6,8-dichloro-2-trifluoromethyl-2H-1-benzopyran-3-carboxylic acid; |
| B-47 | 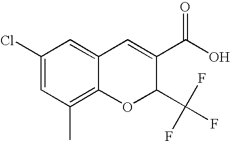 |
| | 6-chloro-8-methyl-2-trifluoromethyl-2H-1-benzopyran-3- |
| | carboxylic acid; |
| B-48 | 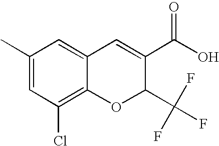 |
| | 8-chloro-6-methyl-2-trifluoromethyl-2H-1-benzopyran-3- |
| | carboxylic acid; |
| B-49 | 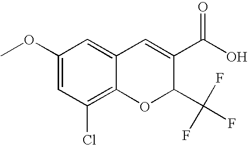 |
| | 8-chloro-6-methoxy-2-trifluoromethyl-2H-1-benzopyran-3- |
| | carboxylic acid; |
| B-50 | 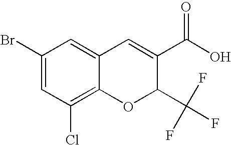 |
| | 6-bromo-8-chloro-2-trifluoromethyl-2H-1-benzopyran-3- |
| | carboxylic acid; |
| B-51 | 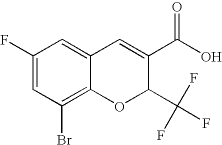 |
| | 8-bromo-6-fluoro-2-trifluoromethyl-2H-1-benzopyran-3- |
| | carboxylic acid; |
| B-52 | 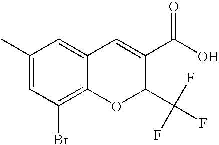 |
| | 8-bromo-6-methyl-2-trifluoromethyl-2H-1-benzopyran-3- |
| | carboxylic acid; |
| B-53 | 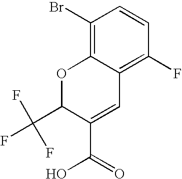 |
| | 8-bromo-5-fluoro-2-trifluoromethyl-2H-1-benzopyran-3- |
| | carboxylic acid; |
| B-54 | 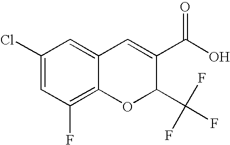 |
| | 6-chloro-8-fluoro-2-trifluoromethyl-2H-1-benzopyran-3- |
| | carboxylic acid; |
| B-55 | 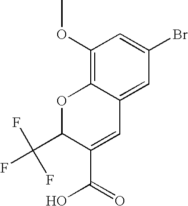 |
| | 6-bromo-8-methoxy-2-trifluoromethyl-2H-1-benzopyran-3- |
| | carboxylic acid; |
| B-56 | 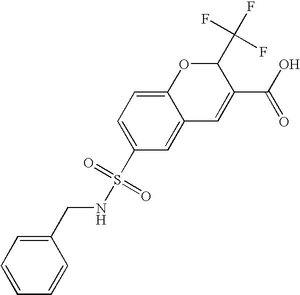 |
| | 6-[[(phenylmethyl)amino]sulfonyl]-2-trifluoromethyl-2H-1- |
| | benzopyran-3-carboxylic acid; |
| B-57 | 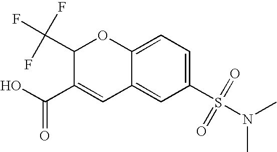 |
| | 6-[(dimethylamino)sulfonyl]-2-trifluoromethyl-2H-1-benzopyran-3- |
| | carboxylic acid; |
| B-58 | 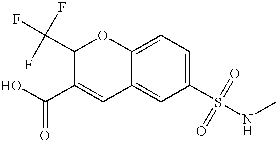 |
| | 6-[(methylamino)sulfonyl]-2-trifluoromethyl-2H-1-benzopyran-3- |
| | carboxylic acid; |
| B-59 | 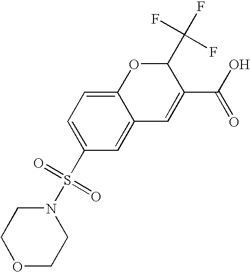 |
| | 6-[(4-morpholino)sulfonyl]-2-trifluoromethyl-2H-1-benzopyran-3- |
| | carboxylic acid; |
| B-60 | 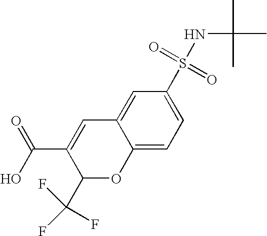 |
| | 1-dimethylethyl)aminosulfonyl]-2-trifluoromethyl- |
| | 2H-1-benzopyran-3-carboxylic acid; |
| B-61 | 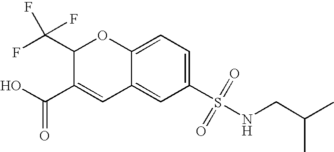 |
| | 6-[(2-methylpropyl)aminosulfonyl]-2-trifluoromethyl-2H-1- |
| | benzopyran-3-carboxylic acid; |
| B-62 | 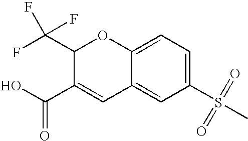 |
| | 6-methylsulfonyl-2-trifluoromethyl-2H-1-benzopyran-3- |
| | carboxylic acid; |
| B-63 | 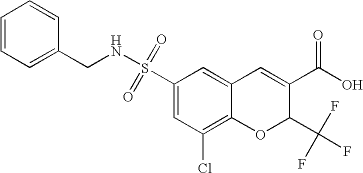 |
| | 8-chloro-6-[[(phenylmethyl)ammo]sulfonyl]-2-trifluoromethyl- |
| | 2H-1-benzopyran-3-carboxylic acid; |
| B-64 | 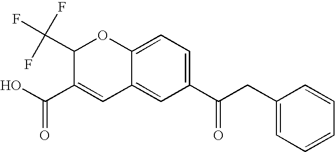 |
| | 6-phenylacetyl-2-trifluoromethyl-2H-1-benzopyran-3- |
| | carboxylic acid; |
| B-65 | 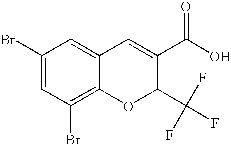 |
| | 6,8-dibromo-2-trifluoromethyl-2H-1-benzopyran-3-carboxylic acid; |
| B-66 | 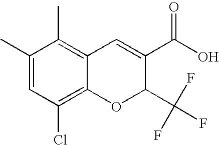 |
| | 8-chloro-5,6-dimethyl-2-trifluoromethyl-2H-1-benzopyran-3- |
| | carboxylic acid; |
| B-67 | 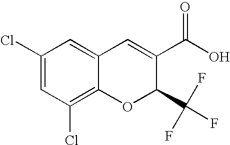 |
| | 6,8-dichloro-(S)-2-trifluoromethyl-2H-1-benzopyran-3- |
| | carboxylic acid; |
| B-68 | 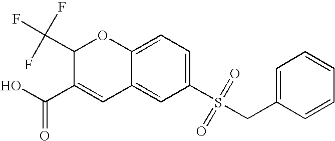 |
| | 6-benzylsulfonyl-2-trifluoromethyl-2H-1-benzopyran-3- |
| | carboxylic acid; |
| B-69 | 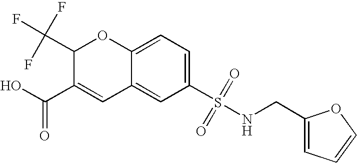 |
| | 6-[[N-(2-furylmethyl)amino]sulfonyl]-2-trifluoromethyl- |
| | 2H-1-benzopyran-3-carboxylic acid; |
| B-70 | 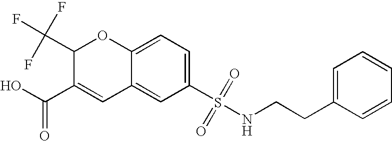 |
| | 6-[[N-(2-phenylethyl)amino]sulfonyl]-2-trifluoromethyl-2H- |
| | 1-benzopyran-3-carboxylic acid; |
| B-71 | 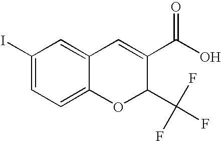 |
| | 6-iodo-2-trifluoromethyl-2H-1-benzopyran-3-carboxylic acid; |
| B-72 | 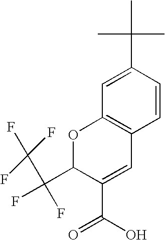 |
| | 7-(1,1-dimethylethyl)-2-pentafluoroethyl-2H- |
| | 1-benzopyran-3-carboxylic acid; |
| B-73 | 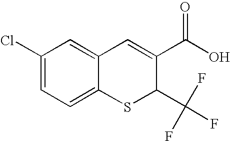 |
| | 6-chloro-2-trifluoromethyl-2H-1-benzothiopyran-3-carboxylic acid; |
| B-74 | 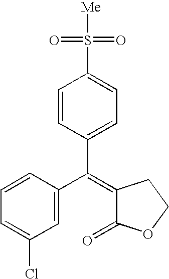 |
| | 3-[(3-chloro-phenyl)-(4-methanesulfonyl-phenyl)-methylene]- |
| | dihydro-furan-2-one or BMS-347070; |
| B-75 | 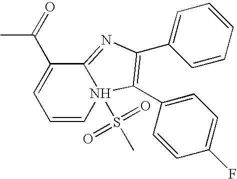 |
| | 8-acetyl-3-(4-fluorophenyl)-2-(4-methylsulfonyl)pbenyl- |
| | imidazo(1,2-a)pyridine; |
| B-76 | 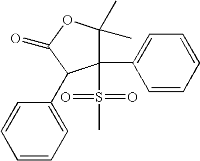 |
| | 5,5-dimethyl-4-(4-methylsulfonyl)phenyl-3-phenyl-2-(5H)- |
| | furanone; |
| B-131 | 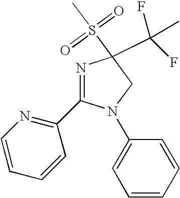 |
| | 2-[1-[4-(methylsulfonyl)phenyl-4-(trifluoromethyl)]-1H- |
| | imidazol-2-yl]pyridine; |
| B-132 | 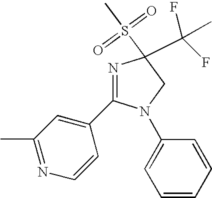 |
| | 2-methyl-4-[1-[4-(methylsulfonyl)phenyl-4-(trifluoromethyl)]- |
| | 1H-imidazol-2-yl]pyridine; |
| B-133 | 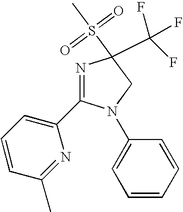 |
| | 2-methyl-6-[1-[4-(methylsulfonyl)phenyl-4-(trifluoromethyl)]- |
| | 1H-imidazol-2-yl]pyridine; |
| B-140 | 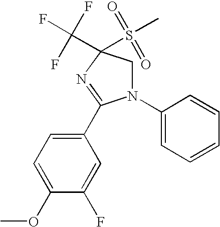 |
| | 2-(3-fluoro-4-methoxyphenyl)-1-[4-(methylsulfonyl)phenyl- |
| | 4-(trifluoromethyl)]-1H-imidazole; |
| B-196 | 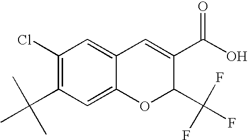 |
| | 6-chloro-7-(1,1-dimethylethyl)-2-trifluoromethyl-2H- |
| | 1-benzopyran-3-carboxylic acid; |
| B-197 | 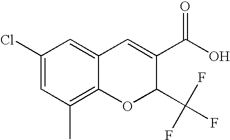 |
| | 6-chloro-8-methyl-2-trifluoromethyl-2H-1-benzopyran-3- |
| | carboxylic acid; |
| B-198 | 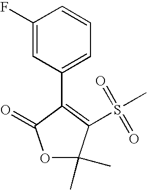 |
| | 5,5-dimethyl-3-(3-fluorophenyl)-4-methylsulfonyl-2(5H)-furanone; |
| B-199 | 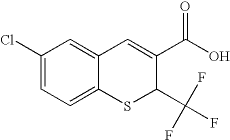 |
| | 6-chloro-2-trifluoromethyl-2H-1-benzothiopyran-3-carboxylic acid; |
| B-211 | 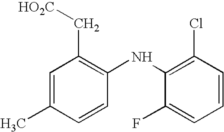 |
| B-212 | 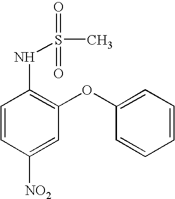 |
| | N-(4-nitro-2-phenoxy-phenyl)-methanesulfonamide |
| B-213 | 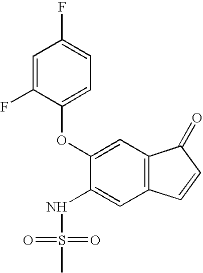 |
| | N-[6-(2,4-difluoro-phenoxy)-1-oxo-inden-5-yl]- |
| | methanesulfonamide |
| B-214 | 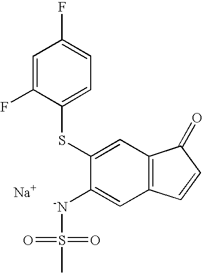 |
| | N-[6-(2,4-difluoro-phenylsulfanyl)-1-oxo-1H-inden-5-yl]- |
| | methanesulfonamide, soldium salt, or L-745337 |
| B-215 | 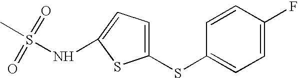 |
| | N-[5-(4-fluoro-phenylsulfanyl)-thiophen-2-yl]- |
| | methanesulfonanude or RWJ-63556 |
| B-217 | 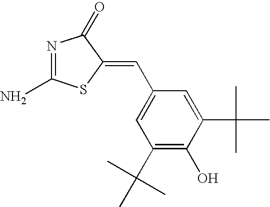 |
| | (5Z)-2-amino-5-[[3,5-bis(1,1-dimethylethyl)-4- |
| | hydroxyphenyl]methylene]-4(5H)-thiazolone or Darbufelone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 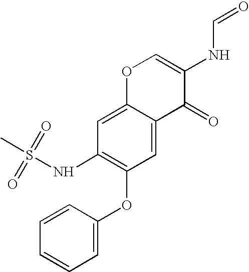 |
| | N-[3-(formylamino)-4-oxo-6-phenoxy-4H-1-benzopyran-7-yl]- |
| | methanesulfonamide or T614 |
| B-225 | D-1367 |
| B-226 | L-748731 |
| B-227 | 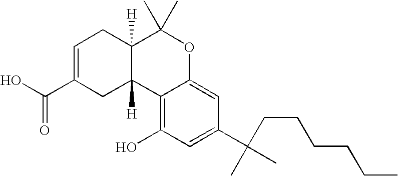 |
| | (6aR,10aR)-3-(1,1-dimethylheptyl)-6a,7,10,10a-tetrahydro-1- |
| | hydroxy-6,6-dimethyl-6H-dibenzo[b,d]pyran-9- |
| | carboxylic acid or CT3 |
| B-228 | CGP-28238 |
| B-229 | 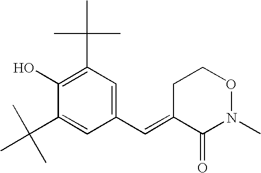 |
| | 4-[[3,5-bis(1,1-dimethylethyl)-4-hydroxyphenyl]methylene] |
| | dihydro-2-methyl-2H-1,2-oxazin-3(4H)-one or BF-389 |
| B-230 | GR-253035 |
| [0266]B-231 | 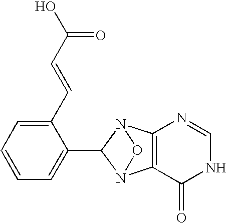 |
| | [0267] 2-(6-dioxo-9H-purin-8-yl)cinnamic acid |
| [0268] | [0269] |
| B-232 | [0270] S-2474 |
| [0271]B-233 | 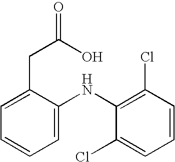 |

| | |
|---|---|
| [0272] | 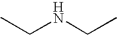 |

The chromene or phenyl acetic acid cyclooxygenase-2 selective inhibitor employed in the present invention can exist in tautomeric, geometric or stereoisomeric forms. Generally speaking, suitable chromene or phenyl acetic acid cyclooxygenase-2 selective inhibitors that are in tautomeric, geometric or stereoisomeric forms are those compounds that inhibit cyclooxygenase-2 activity by about 25%, more typically by about 50%, and even more typically, by about 75% or more when present at a concentration of 100 μM or less. The present invention contemplates all such compounds, including cis- and trans-geometric isomers, E- and Z-geometric isomers, R— and S-enantiomers, diastereomers, d-isomers, l-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present.

The chromene or phenyl acetic acid cyclooxygenase-2 selective inhibitors utilized in the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

The chromene or phenyl acetic acid cyclooxygenase-2 selective inhibitors useful in the practice of the present invention can be formulated into pharmaceutical compositions and administered by a number of suitable means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the chromene or phenyl acetic acid cyclooxygenase-2 selective inhibitor will vary depending upon the patient and the particular mode of administration. In general, the pharmaceutical compositions may contain a chromene or phenyl acetic acid cyclooxygenase-2 selective inhibitor in the range of about 0.1 to 2000 mg, more typically, in the range of about 0.5 to 500 mg and still more typically, between about 1 and 200 mg. A daily dose of about 0.01 to 100 mg/kg body weight, or more typically, between about 0.1 and about 50 mg/kg body weight and even more typically, from about 1 to 20 mg/kg body weight, may be appropriate. The daily dose can be administered in one to about four doses per day.

Those skilled in the art will appreciate that dosages may also be determined with guidance from Goodman & Goldman's The Pharmacological Basis of Therapeutics, Ninth Edition (1996), Appendix II, pp.1707-1711 and from Goodman & Goldman's The Pharmacological Basis of Therapeutics, Tenth Edition (2001), Appendix II, pp. 475493.

### Ace Inhibitors

In addition to a chromene or phenyl acetic acid cyclooxygenase-2 selective inhibitor, the composition of the invention also comprises a therapeutically effective amount of an ACE inhibitor or an isomer, a pharmaceutically acceptable salt, ester, or prodrug thereof. A number of ACE inhibitors may be employed in the present invention. In some aspects, the ACE inhibitor may reverse or lessen central nervous system cell damage following a reduction in blood flow to the central nervous system. In other aspects, the ACE inhibitor may reverse or lessen central nervous system cell damage following a traumatic brain or spinal cord injury.

A number of different suitable ACE inhibitors, or isomers, pharmaceutically acceptable salts, esters, or prodrugs thereof may be employed in the present invention. In one embodiment, the ACE inhibitor is benazepril (marketed under the trademark Lotensin®). In another embodiment, the ACE inhibitor is captopril (marketed under the trademark Captoten®). In still another embodiment, the ACE inhibitor is enalapril (marketed under the trademark Vasotec®). In yet another embodiment, the ACE inhibitor is fosinopril (marketed under the trademark Monopril®). In a further embodiment, the ACE inhibitor is lisinopril (marketed under the trademark Prinivil®). In still another embodiment, the ACE inhibitor is moexipril (marketed under the trademark Univasc®). In yet another embodiment, the ACE inhibitor is perindopril (marketed under the trademark Aceon®). In a further embodiment, the ACE inhibitor is quinapril (marketed under the trademark Accuprilo). In still another embodiment, the ACE inhibitor is ramipril (marketed under the trademark Altace®). In yet another embodiment, the ACE inhibitor is trandolapril (marketed under the trademark Mavik®). Other suitable ACE inhibitors are shown in Table 4 below.

TABLE 4
EXAMPLES OF ACE INHIBITORS

| COMPOUND | |
|---|---|
| NUMBER | STRUCTURE |
| D-1 | 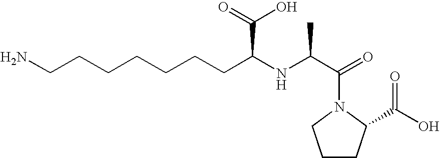 |
| | 1-[N-(8-amino-1-carboxyoctyl-L- |
| | alanyl]-(S)-L-Proline |
| D-2 | 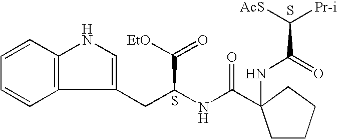 |
| | N-[[1-[[2-(acetylthio)-3-methyl-1-oxobutyl]amino] |
| | cyclopentyl]carbonyl]-ethyl ester(S)-L- |
| | Tryptophan |
| D-3 | 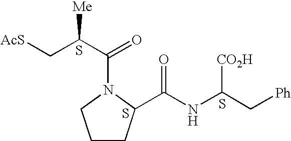 |
| | 1-[(2S)-3-(acetylthio)-2-methyl-1- |
| | oxopropyl]-L-prolyl-L-Phenylalanine |
| D-4 | 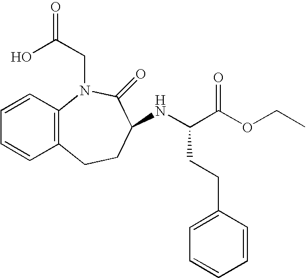 |
| | 3-[[(1S)-1-(ethoxycarbonyl)-3-phenylpropyl]amino]- |
| | 2,3,4,5-tetrahydro-2-oxo-(3S)-1H-1-Benzazepine-1- |
| | acetic acid |
| D-5 | 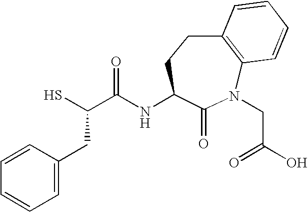 |
| | [S-(R*,R*)]-2,3,4,5-tetrahydro-3-[(2-mercapto- |
| | 1-oxo-3-phenylpropyl)amino]-2-oxo-1H- |
| | benzazepine-1-acetic acid |
| D-6 | 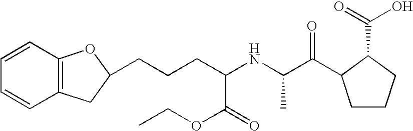 |
| | N-[4-(2,3-dihydro-2-benzofuranyl)-1- |
| | (ethoxycarbonyl)butyl]-L-alanyl-L-Proline |

| | |
|---|---|
| D-7 | PAGE 1-A |

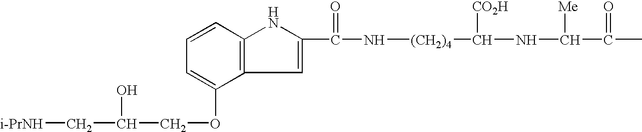

PAGE 1-B

| | 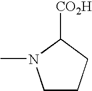 |
|---|---|
| | N-[(S) -1-carboxy-5-(4-[(S)-2-hydroxy-3- |
| | isopropylaminoproproxy]-1H-indole-2- |
| | carboxamido)pentyl]-(S)-alanyl-(S)-proline |
| D-8 | 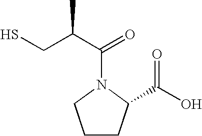 |
| | 1-[(2S)-3-Mercapto-2-methyl-1- |
| | oxopropyl]-L-proline |
| D-9 | 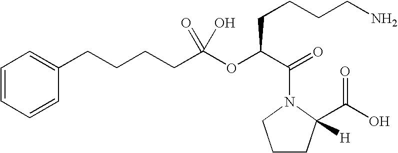 |
| | 1-[6-amino-2-[[hydroxy(4-phenylbutyl) |
| | phosphinyl]oxy]-1-oxohexyl]-(S)-L-Proline |
| D-10 | 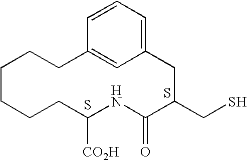 |
| | (3S,6S)-4-Oxo-3-(sulfanylmethyl)-5- |
| | azabicyclo[10.3.1]hexadeca-1(16),12,14-triene-6- |
| | carboxylic acid |
| D-11 | 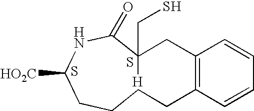 |
| | 2,3,4,5,6,7,8,9-octahydro-2-(mercaptomethyl)-3-oxo- |
| | (2S,5S)-1H-4-Benzazacycloundecine-5-carboxylic acid |
| D-12 | 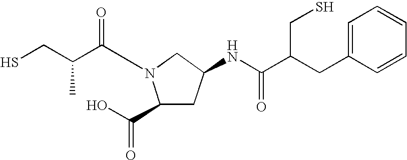 |
| D-13 | 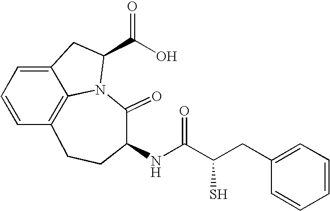 |
| | 10-[(3-Phenyl-2-mercapto-1-oxo)propylamino]- |
| | 9-oxo-8-azatricyclo[4.3.2]-1,3,5- |
| | hexahydrododecan-7-carboxylic acid |
| D-14 | 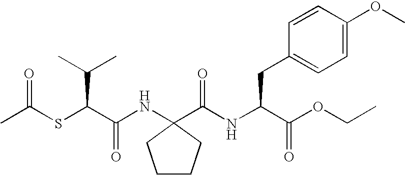 |
| | N-[1-[2(S)-(Acetylsulfanyl)-3-methylbutyramido] |
| | cyclopent-1-ylcarbonyl]-4-O-methyl-L-tyrosine |
| | ethyl ester |
| D-15 | 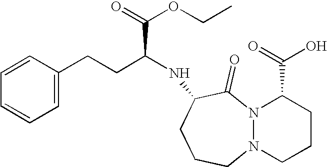 |
| | 9-[[(1S)-1-(ethoxycarbonyl)-3- |
| | phenylpropyl]amino]octahydro-10-oxo-(1S,9S)-6H- |
| | Pyridazino[1,2-a][1,2]diazepine-1-carboxylic acid |
| D-16 | 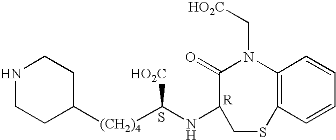 |
| | 3-[[1-carboxy-5-(4-piperidinyl) pentyl]amino]- |
| | 3,4,dihydro-4-oxo[s-(R*,S*)]-1,5-Benzothiazepine- |
| | 5(2H)-acetic acid |
| D-17 | 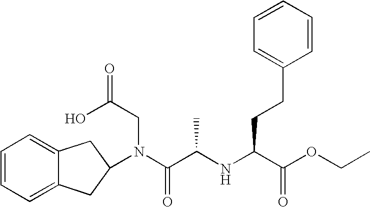 |
| | N-(2,3-dihydro-1H-inden-2-yl)-N-[N-[1- |
| | (ethoxycarbonyl)-3-phenylpropyl]-L-alanyl]-(S)- |
| | Glycine |
| D-18 | 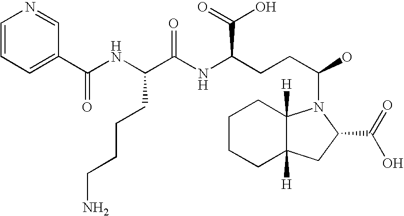 |
| | (2S,3aS,7aS)-1-[N-(3-Pyridylcarbonyl)-1-lysyl- |
| | gamma-dglutamyl]octahydroindole-2-carboxylic acid |
| D-19 | 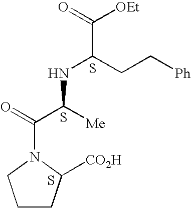 |
| | N-[(1S)-1-(ethoxycarbonyl)-3-phenylpropyl]-L-alanyl-L- |
| | Proline |
| D-20 | 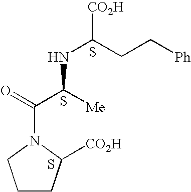 |
| | N-[(1S)-1-carboxy-3-phenylpropyl]-L-alanyl-L-Proline |
| D-21 | No Structure Available |
| | (S)-N-[N-[1-[ethoxycarbonyl]-3-phenylpropyl]-L- |
| | alanyl]-N-[2-ethoxyethoxy]-glycine |
| D-22 | 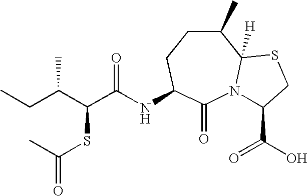 |
| | 3R,6S,9R,9aR)-6-[(2S,3S)-2-acetylthio-3- |
| | methylpentylamido]-9-methyl-5- |
| | oxooctahydroazepino[2,1-b]thiazole-3-carboxylic |
| | acid |
| D-23 | 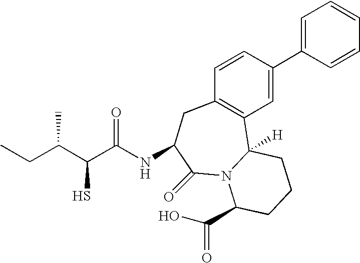 |
| | 1,2,3,4,6,7,8,12b-octahydro-7-[(2-mercapto-3-methyl-1- |
| | oxopentyl)amino]-6-oxo-11-phenyl-[4S- |
| | [4a,7a(2R*,3R*)12bb]]-Pyrido[2,1-a][2]benzazepine-4- |
| | carboxylic acid |
| D-24 | 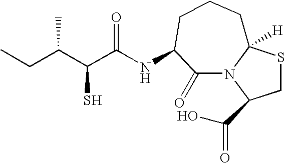 |
| | octahydro-6-[((2-mercapto-3-methyl-1- |
| | oxopentyl)amino)]-5-oxo-[(3R-[(3alpha,6alpha |
| | (2S*,3S*)9alphabeta)])]-Thiazolo[(3,2-a)]azepine- |
| | 3-carboxylic acid |
| D-25 | 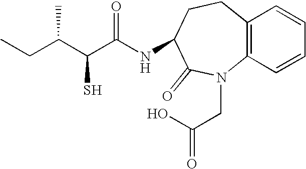 |
| D-26 | 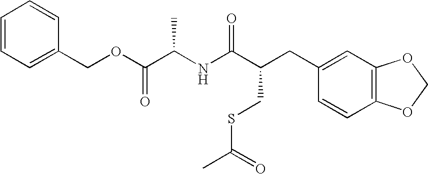 |
| | (−)-(S)-N-[2-Acetylthiomethyl)-3-(3,4- |
| | methylenedioxyphenyl)propanoyl]-(S)-alanine benzyl |
| | ester |
| D-27 | 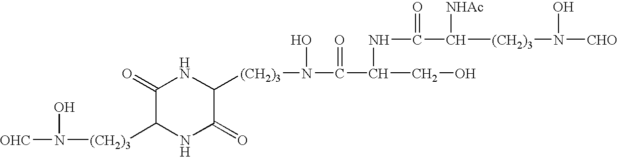 |
| | N2-acetyl-N5-formyl-N5-hydroxy-L-omithyl-N-[3- |
| | [(2S,5R)-5-[3-(formylhydroxyamino)propyl]-3,6-dioxo-2- |
| | piperazinyl]propyl]-N-hydroxy-L-Serinamide |
| D-28 | 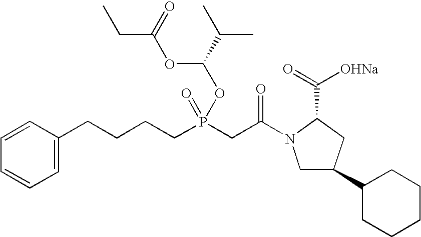 |
| | 4-cyclohexyl-1-[[[2-methyl-1-(1-oxopropoxy) |
| | propoxy](4-phenylbutyl)phosphinyl]acetyl]-[1[S*(R*)], |
| | 2alpha,4beta]-L-Proline, sodium salt |
| D-29 | 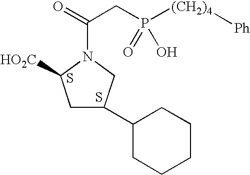 |
| | [4(S)-Cyclohexyl-2(R)-I-prolin-1yl]carbonylmethyl- |
| | (4-phenylbutyl) phosphinic acid |
| D-30 | 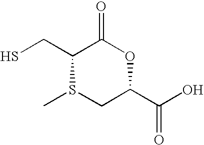 |
| | 2(R)-[3-Mercapto-2(S)-methylpropranoyloxy]-3- |
| | (methylthio)propanoic acid |
| D-31 | 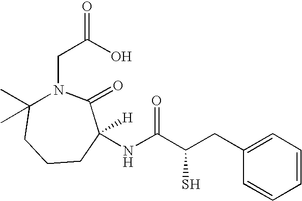 |
| | hexahydro-6-[[[2S]-2-mercapto-1-oxo-3- |
| | phenylpropyl]amino]-2,2-dimethyl-7-oxo-[6S]-1H- |
| | 1-acetic acid |
| D-32 | 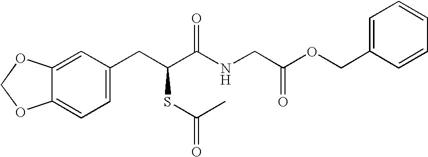 |
| | N-[3-(acetylthio)-2-(1,3-benzodioxol-5-ylmethyl)- |
| | 1-oxopropyl]-phenylmethyl ester,(5)-Glycine |
| D-33 | 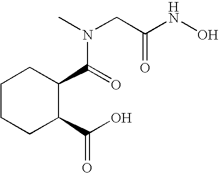 |
| | 2-[[[2-(hydroxyamino)-2-oxoethyl]-N- |
| | methylamino]carbonyl]-(+)-cis-(1S,2R)- |
| | Cyclohexanecarboxylic acid |
| D-34 | 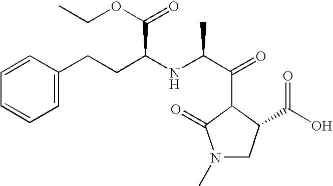 |
| | 3-[2-[[1-(ethoxycarbonyl)-3-phenylpropyl]amino]- |
| | 1-oxopropyl]-1-methyl-2-oxo-, |
| | monohydrochloride[4S-[3[R*(R*)],4R*]]-4- |
| | Imidazolidinecarboxylic acid |
| D-35 | 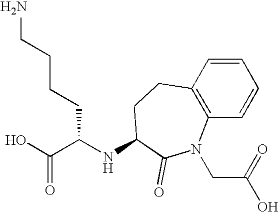 |
| | 3-[(5-amino-1-carboxypentyl)amino]-2,3,4,5- |
| | tetrahydro-2-oxo-dihydrobromide [S-(R*,R*)]- |
| | 1H-1-benzazepine-1-acetic acid |
| D-36 | 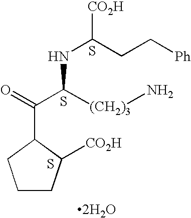 |
| | N2-[(1S)-1-carboxy-3-phenylpropyl]-L-lysyl-L-Proline, |
| | dihydrate |
| D-37 | 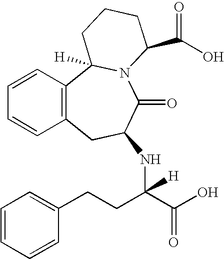 |
| | 7-[(1-carboxy-3-phenylpropyl)amino]- |
| | 1,2,3,4,6,7,8,12a-octahydro-6-oxo-[4S- |
| | [4alpha,7alpha,(R*),12bbeta]]-Pyrido[2,1-a][2] |
| | benzazepine-4-carboxylic acid |
| D-38 | 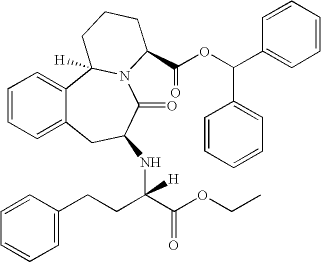 |
| | 4(S)-4alpha,7alpha(R*),12bbeta]-7-[1- |
| | (Ethoxycarbonyl)-3-phenylpropylamino]- |
| | 1,2,3,4,5,6,7,8,12b-octahydro-6-oxopyrido[2,1-a] |
| | [2]benzazepine-4-carboxylic acid diphenylmethyl |
| | ester |
| D-39 | 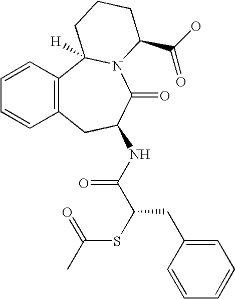 |
| | (4S,7S,12bR)-7-[2(S)-(Acetylthio)-3- |
| | phenylpropionamido]-6-oxo-1,2,3,4,6,7,8,12b- |
| | octahydropyrido[2,1-a][2]benzazepine-4-carboxylic |
| | acid |
| D-40 | 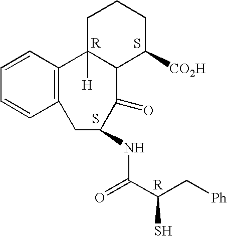 |
| | 6-Oxo-7(S)-[3-phenyl-2(R)- |
| | sulfanylpropanamido]-6-oxo-1,2,3,4,6,7,8,12b(R)- |
| | octahydropyrido[2,1-a]-2-benzazepine-4(S)- |
| | carboxylic acid |
| D-41 | 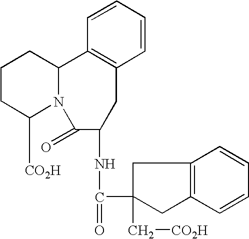 |
| | 7-[[[2-(carboxymethyl)-2,3-dihydro-1H-inden-2- |
| | yl]carbonyl]amino]-1,2,3,4,6,7,8,12b-octahydro-6-oxo- |
| | (4S,7S,12bR)-Pyrido[2,1-a][2]benzazepine-4-carboxylic |
| | acid |
| D-42 | 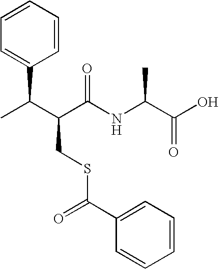 |
| | N-[(2S,3R)-2-benzoylthiomethyl-1-oxo-3- |
| | phenylbutyl]-(S)-Alanine |
| D-43 | 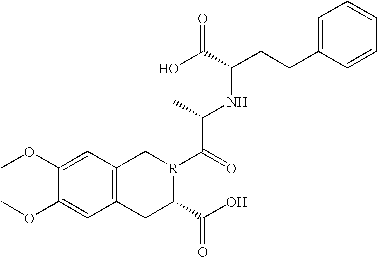 |
| | 2-[2-[[1-(ethoxycarbonyl)-3-phenylpropyl]amino]- |
| | 1-oxopropyl]-1,2,3,4-tetrahydro-6,7-dimethoxy- |
| | [3S-[2[R*(R*)13R*]]-3-Isoquinoline carboxylic acid, |
| | monohydrochloride |
| D-44 | 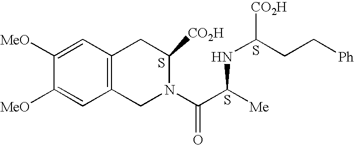 |
| | 2-[(2S)-2-[[(1S)-1-carboxy-3-phenylpropyl]amino]-1- |
| | oxopropyl]-1,2,3,4-tetrahydro-6,7-dimethoxy-(3S)-3- |
| | lsoquinolinecarboxylic acid |
| D-45 | 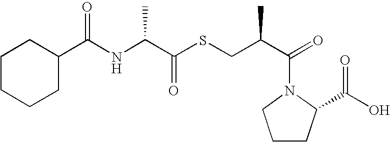 |
| | 1-[3-[[2-[(cyclohexylcarbonyl)amino]-1- |
| | oxopropyl]thio]-2-methyl-1-oxopropyl]-calcium salt |
| | (2:1)[R-(R*,S*)]-L-Proline |
| D-46 | 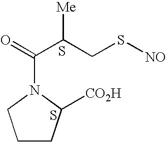 |
| | 1-[2-methyl-3-(nitrosothio)-1-oxopropyl]-,(S)-L- |
| | Proline |
| D-47 | 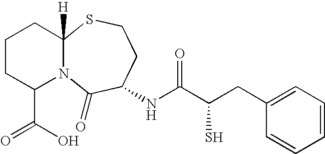 |
| | (4S,7S,10aS)-Octahydro-4-[(5)-alpha- |
| | mercaptohydrocinnamido]-5-oxo-7H-pyrido[2,1- |
| | b][1,3]thiazepine-7-carboxylic acid |
| D-48 | 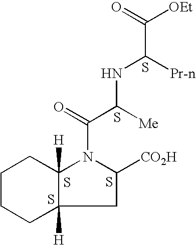 |
| | 1-[(2S)-2-[[(1S)-1-(ethoxycarbonyl)butyl]amino]-1- |
| | oxopropyl]octahydro-(2S,3aS,7aS)-1H-Indole-2- |
| | carboxylic acid |

Generally speaking, the pharmacokinetics of the particular agent to be administered will dictate the most preferred method of administration and dosing regiment. The ACE inhibitor can be administered as a pharmaceutical composition with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates (such as lactose, sucrose, dextrose, and mannose), albumin, starch, cellulose, silica gel, polyethylene glycol (PEG), dried skim milk, rice flour, magnesium stearate, and the like. Suitable formulations and additional carriers are described in Remington's Pharmaceutical Sciences, (17.sup.th Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation.

Moreover, the ACE inhibitor can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharin, cellulose, or magnesium carbonate.

In another embodiment, the ACE inhibitor can be administered intravenously, parenterally, intramuscular, subcutaneously, orally, nasally, topically, by inhalation, by implant, by injection, or by suppository. For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used. Pulmonary administration can be accomplished, for example, using any of various delivery devices known in the art such as an inhaler. See. e.g. S. P. Newman (1984) in Aerosols and the Lung, Clarke and Davis (eds.), Butterworths, London, England, pp.197-224; PCT Publication No. WO 92/16192; PCT Publication No. WO 91/08760. For parenteral application, particularly suitable are injectable, sterile solutions, preferably oily or aqueous solutions, as well as suspensions, emulsions, or implants. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like.

The actual effective amounts of compound or drug can and will vary according to the specific composition being utilized, the mode of administration and the age, weight and condition of the subject. Dosages for a particular individual subject can be determined by one of ordinary skill in the art using conventional considerations. But in general, the amount of ACE inhibitor will be between about 0.5 to about 750 milligrams per day. The daily dose can be administered in one to four doses per day.

In one embodiment, when the ACE inhibitor comprises benazepril, typically the amount administered is within a range of from about 5 to about 400 milligrams per day, and even more typically, between about 10 to about 200 milligrams per day.

In another embodiment, when the ACE inhibitor is enalapril, typically the amount administered is within a range of from about 1.0 to about 50 milligrams per day, and even more typically, between about 2.5 to about 40 milligrams per day.

In yet another embodiment, when the ACE inhibitor is fosinopril, generally the amount administered is within a range of from about 5 to about 50 milligrams per day, and even more typically, between about 10 to about 30 milligrams per day.

In still another embodiment, when the ACE inhibitor is lisinopril, typically the amount administered is within a range of from about 5 to about 50 milligrams per day, and even more typically, between about 10 to about 30 milligrams per day.

In yet a further embodiment, when the ACE inhibitor is moexipril, typically the amount administered is within a range of from about 5 to about 50 milligrams per day, and even more typically, between about 10 to about 30 milligrams per day.

In still another embodiment, when the ACE inhibitor is ramipril, typically the amount administered is within a range of from about 1 to about 40 milligrams per day, and even more typically, between about 2.5 to about 20 milligrams per day.

When the composition is employed to treat a vaso-occlusive event, the timing of the administration of the ACE inhibitor before or after the onset of the vaso-occlusive event will vary considerably depending upon the particular vaso-occlusive event being treated. Generally speaking, the ACE inhibitor is preferably administered to the subject immediately after the onset of the vaso-occlusive event. By way of example, if the vaso-occlusive event is an acute myocardial infarction (AMI), the ACE inhibitor is typically administered to the subject within 24 hours of the onset of symptoms of the AMI. More typically, the ACE inhibitor is administered within about 0 to 12 hours of the onset of symptoms of the AMI. Even more typically, the ACE inhibitor is administered within about 0 to 6 hours of the onset of symptoms of the AMI. By way of further example, if the vaso-occlusive event is an acute ischemic stroke, typically the ACE inhibitor is administered within about 0-4 hours after the onset of symptoms of the acute ischemic stroke. Even more typically, the selective ACE inhibitor is administered within about 0 to 2 hours after the onset of the symptoms of the acute ischemic stroke. Still more typically, the ACE inhibitor is administered within about 0 to 1 hour after the onset of the symptoms of the acute ischemic stroke.

Moreover, the timing of the administration of the chromene or phenyl acetic acid cyclooxygenase-2 selective inhibitor in relation to the administration of the ACE inhibitor may also vary from subject to subject and depend upon the condition being treated. In one embodiment of the invention, the chromene or phenyl acetic acid cyclooxygenase-2 selective inhibitor and ACE inhibitor may be administered substantially simultaneously, meaning that both agents may be administered to the subject at approximately the same time. For example, the chromene or phenyl acetic acid cyclooxygenase-2 selective inhibitor or pharmaceutically acceptable salt or prodrug thereof is administered during a continuous period beginning on the same day as the beginning of the ACE inhibitor and extending to a period after the end of the ACE inhibitor. Alternatively, the chromene or phenyl acetic acid cyclooxygenase-2 selective inhibitor and ACE inhibitor may be administered sequentially, meaning that they are administered at separate times during separate treatments. In one embodiment, for example, the chromene or phenyl acetic acid cyclooxygenase-2 selective inhibitor or, an isomer, ester, a pharmaceutically acceptable salt or prodrug thereof is administered during a continuous period beginning prior to administration of the ACE inhibitor and ending after administration of the ACE inhibitor. Of course, it is also possible that the chromene or phenyl acetic acid cyclooxygenase-2 selective inhibitor may be administered either more or less frequently than the ACE inhibitor. One skilled in the art can readily design suitable treatment regiments for a particular subject depending on the particular vaso-occlusive event being treated. Moreover, it will be apparent to those skilled in the art that it is possible, and perhaps desirable, to combine various times and methods of administration in the practice of the present invention.

### Combination Therapies

Generally speaking, it is contemplated that the composition employed in the practice of the invention may include one or more of any of the cyclooxygenase-2 selective inhibitors detailed above in combination with one or more of any of the ACE inhibitors detailed above. By way of a non-limiting example, Table 5a details a number of suitable combinations that are useful in the methods and compositions of the current invention. The combination may also include an isomer, a pharmaceutically acceptable salt, ester, or prodrug of any of the cyclooxygenase-2 selective inhibitors and/or ACE inhibitors listed in Table 5a.

TABLE 5a

| Cyclooxygenase-2 Selective | |
|---|---|
| Inhibitor | ACE inhibitor |
| a compound having formula I | Benazepril |
| a compound having formula I | Enalapril |
| a compound having formula I | Fosinopril |
| a compound having formula I | Lisinopril |
| a compound having formula I | Moexipril |
| a compound having formula I | Perindopril |
| a compound having formula I | Quinapril |
| a compound having formula I | Ramipril |
| a compound having formula I | Trandolapril |
| a compound having formula III | Benazepril |
| a compound having formula III | Enalapril |
| a compound having formula III | Fosinopril |
| a compound having formula III | Lisinopril |
| a compound having formula III | Moexipril |
| a compound having formula III | Perindopril |
| a compound having formula III | Quinapril |
| a compound having formula III | Ramipril |
| a compound having formula III | Trandolapril |
| a compound having formula IV | Benazepril |
| a compound having formula IV | Enalapril |
| a compound having formula IV | Fosinopril |
| a compound having formula IV | Lisinopril |
| a compound having formula IV | Moexipril |
| a compound having formula IV | Perindopril |
| a compound having formula IV | Quinapril |
| a compound having formula IV | Ramipril |
| a compound having formula IV | Trandolapril |
| a compound having formula V | Benazepril |
| a compound having formula V | Enalapril |
| a compound having formula V | Fosinopril |
| a compound having formula V | Lisinopril |
| a compound having formula V | Moexipril |
| a compound having formula V | Perindopril |
| a compound having formula V | Quinapril |
| a compound having formula V | Ramipril |
| a compound having formula V | Trandolapril |

By way of further example, Table 5b details a number of suitable combinations that may be employed in the methods and compositions of the present invention. The combination may also include an isomer, a pharmaceutically acceptable salt, ester, or prodrug of any of the cyclooxygenase-2 selective inhibitors and/or ACE inhibitors listed in Table 5b.

TABLE 5b

| Cyclooxygenase-2 Selective Inhibitor | ACE inhibitor |
|---|---|
| a compound selected from the group consisting | Benazepril |
| of B-3, B-4, B-5, B-6, B7, B-8, B-9, B-10, B-11, B- | |
| 12, B-13, B-14, B-15, B-16, B-17, B-27, B-28, B- | |
| 29, B-30, B-31, B-32, B-33, B-34, B-35, B-36, B- | |
| 37, B-38, B-39, B-40, B-41, B-42, B-43, B-44, B- | |
| 45, B-46, B-47, B-48, B-49, B-50, B-51, B-52, B- | |
| 53, B-54, B-55, B-56, B-57, B-58, B-59, B-60, B- | |
| 61, B-62, B-63, B-64, B-65, B-66, B-67, B-68, B- | |
| 69, B-70, B-71, B-72, B-73. B-74, B-75, B-76, B- | |
| 131, B-132, B-133, B-140, B-196, B-197, B-198, | |
| B-199, B-211, B-212, B-213, B-214, B-215, B- | |
| 217, B-218, B-219, B-220, B-221, B-222, B-223, | |
| B-224, B-225, B-225, B-227, B-228, B-229, B- | |
| 230, B-231, B-232, and B-233. | |
| a compound selected from the group consisting | Enalapril |
| of B-3, B-4, B-5, B-6, B7, B-8, B-9, B-10, B-11, B- | |
| 12, B-13, B-14, B-15, B-16, B-17, B-27, B-28, B- | |
| 29, B-30, B-31, B-32, B-33, B-34, B-35, B-36, B- | |
| 37, B-38, B-39, B-40, B-41, B-42, B-43, B-44, B- | |
| 45, B-46, B-47, B-48, B-49, B-50, B-51, B-52, B- | |
| 53, B-54, B-55, B-56, B-57, B-58, B-59, B-60, B- | |
| 61, B-62, B-63, B-64, B-65, B-66, B-67, B-68, B- | |
| 69, B-70, B-71, B-72, B-73. B-74, B-75, B-76, B- | |
| 131, B-132, B-133, B-140, B-196, B-197, B-198, | |
| B-199, B-211, B-212, B-213, B-214, B-215, B- | |
| 217, B-218, B-219, B-220, B-221, B-222, B-223, | |
| B-224, B-225, B-225, B-227, B-228, B-229, B- | |
| 230, B-231, B-232, and B-233. | |
| a compound selected from the group consisting | Fosinopril |
| of B-3, B-4, B-5, B-6, B7, B-8, B-9, B-10, B-11, B- | |
| 12, B-13, B-14, B-15, B-16, B-17, B-27, B-28, B- | |
| 29, B-30, B-31, B-32, B-33, B-34, B-35, B-36, B- | |
| 37, B-38, B-39, B-40, B-41, B-42, B-43, B-44, B- | |
| 45, B-46, B-47, B-48, B-49, B-50, B-51, B-52, B- | |
| 53, B-54, B-55, B-56, B-57, B-58, B-59, B-60, B- | |
| 61, B-62, B-63, B-64, B-65, B-66, B-67, B-68, B- | |
| 69, B-70, B-71, B-72, B-73. B-74, B-75, B-76, B- | |
| 131, B-132, B-133, B-140, B-196, B-197, B-198, | |
| B-199, B-211, B-212, B-213, B-214, B-215, B- | |
| 217, B-218, B-219, B-220, B-221, B-222, B-223, | |
| B-224, B-225, B-225, B-227, B-228, B-229, B- | |
| 230, B-231, B-232, and B-233. | |
| a compound selected from the group consisting | Lisinopril |
| of B-3, B-4, B-5, B-6, B7, B-8, B-9, B-10, B-11, B- | |
| 12, B-13, B-14, B-15, B-16, B-17, B-27, B-28, B- | |
| 29, B-30, B-31, B-32, B-33, B-34, B-35, B-36, B- | |
| 37, B-38, B-39, B-40, B-41, B-42, B-43, B-44, B- | |
| 45, B-46, B-47, B-48, B-49, B-50, B-51, B-52, B- | |
| 53, B-54, B-55, B-56, B-57, B-58, B-59, B-60, B- | |
| 61, B-62, B-63, B-64, B-65, B-66, B-67, B-68, B- | |
| 69, B-70, B-71, B-72, B-73. B-74, B-75, B-76, B- | |
| 131, B-132, B-133, B-140, B-196, B-197, B-198, | |
| B-199, B-211, B-212, B-213, B-214, B-215, B- | |
| 217, B-218, B-219, B-220, B-221, B-222, B-223, | |
| B-224, B-225, B-225, B-227, B-228, B-229, B- | |
| 230, B-231, B-232, and B-233. | |
| a compound selected from the group consisting | Moexipril |
| of B-3, B-4, B-5, B-6, B7, B-8, B-9, B-10, B-11, B- | |
| 12, B-13, B-14, B-15, B-16, B-17, B-27, B-28, B- | |
| 29, B-30, B-31, B-32, B-33, B-34, B-35, B-36, B- | |
| 37, B-38, B-39, B-40, B-41, B-42, B-43, B-44, B- | |
| 45, B-46, B-47, B-48, B-49, B-50, B-51, B-52, B- | |
| 53, B-54, B-55, B-56, B-57, B-58, B-59, B-60, B- | |
| 61, B-62, B-63, B-64, B-65, B-66, B-67, B-68, B- | |
| 69, B-70, B-71, B-72, B-73. B-74, B-75, B-76, B- | |
| 131, B-132, B-133, B-140, B-196, B-197, B-198, | |
| B-199, B-211, B-212, B-213, B-214, B-215, B- | |
| 217, B-218, B-219, B-220, B-221, B-222, B-223, | |
| B-224, B-225, B-225, B-227, B-228, B-229, B- | |
| 230, B-231, B-232, and B-233. | |
| a compound selected from the group consisting | Perindopril |
| of B-3, B-4, B-5, B-6, B7, B-8, B-9, B-10, B-11, B- | |
| 12, B-13, B-14, B-15, B-16, B-17, B-27, B-28, B- | |
| 29, B-30, B-31, B-32, B-33, B-34, B-35, B-36, B- | |
| 37, B-38, B-39, B-40, B-41, B-42, B-43, B-44, B- | |
| 45, B-46, B-47, B-48, B-49, B-50, B-51, B-52, B- | |
| 53, B-54, B-55, B-56, B-57, B-58, B-59, B-60, B- | |
| 61, B-62, B-63, B-64, B-65, B-66, B-67, B-68, B- | |
| 69, B-70, B-71, B-72, B-73. B-74, B-75, B-76, B- | |
| 131, B-132, B-133, B-140, B-196, B-197, B-198, | |
| B-199, B-211, B-212, B-213, B-214, B-215, B- | |
| 217, B-218, B-219, B-220, B-221, B-222, B-223, | |
| B-224, B-225, B-225, B-227, B-228, B-229, B- | |
| 230, B-231, B-232, and B-233. | |
| a compound selected from the group consisting | Quinapril |
| of B-3, B-4, B-5, B-6, B7, B-8, B-9, B-10, B-11, B- | |
| 12, B-13, B-14, B-15, B-16, B-17, B-27, B-28, B- | |
| 29, B-30, B-31, B-32, B-33, B-34, B-35, B-36, B- | |
| 37, B-38, B-39, B-40, B-41, B-42, B-43, B-44, B- | |
| 45, B-46, B-47, B-48, B-49, B-50, B-51, B-52, B- | |
| 53, B-54, B-55, B-56, B-57, B-58, B-59, B-60, B- | |
| 61, B-62, B-63, B-64, B-65, B-66, B-67, B-68, B- | |
| 69, B-70, B-71, B-72, B-73. B-74, B-75, B-76, B- | |
| 131, B-132, B-133, B-140, B-196, B-197, B-198, | |
| 217, B-218, B-219, B-220, B-221, B-222, B-223, | |
| B-224, B-225, B-225, B-227, B-228, B-229, B- | |
| 230, B-231, B-232, and B-233. | |
| a compound selected from the group consisting | Ramipril |
| of B-3, B-4, B-5, B-6, B7, B-8, B-9, B-10, B-11, B- | |
| 12, B-13, B-14, B-15, B-16, B-17, B-27, B-28, B- | |
| 29, B-30, B-31, B-32, B-33, B-34, B-35, B-36, B- | |
| 37, B-38, B-39, B-40, B-41, B-42, B-43, B-44, B- | |
| 45, B-46, B-47, B-48, B-49, B-50, B-51, B-52, B- | |
| 53, B-54, B-55, B-56, B-57, B-58, B-59, B-60, B- | |
| 61, B-62, B-63, B-64, B-65, B-66, B-67, B-68, B- | |
| 69, B-70, B-71, B-72, B-73. B-74, B-75, B-76, B- | |
| 131, B-132, B-133, B-140, B-196, B-197, B-198, | |
| B-199, B-211, B-212, B-213, B-214, B-215, B- | |
| 217, B-218, B-219, B-220, B-221, B-222, B-223, | |
| B-224, B-225, B-225, B-227, B-228, B-229, B- | |
| 230, B-231, B-232, and B-233. | |
| a compound selected from the group consisting | Trandolapril |
| of B-3, B-4, B-5, B-6, B7, B-8, B-9, B-10, B-11, B- | |
| 12, B-13, B-14, B-15, B-16, B-17, B-27, B-28, B- | |
| 29, B-30, B-31, B-32, B-33, B-34, B-35, B-36, B- | |
| 37, B-38, B-39, B-40, B-41, B-42, B-43, B-44, B- | |
| 45, B-46, B-47, B-48, B-49, B-50, B-51, B-52, B- | |
| 53, B-54, B-55, B-56, B-57, B-58, B-59, B-60, B- | |
| 61, B-62, B-63, B-64, B-65, B-66, B-67, B-68, B- | |
| 69, B-70, B-71, B-72, B-73. B-74, B-75, B-76, B- | |
| 131, B-132, B-133, B-140, B-196, B-197, B-198, | |
| B-199, B-211, B-212, B-213, B-214, B-215, B- | |
| 217, B-218, B-219, B-220, B-221, B-222, B-223, | |
| B-224, B-225, B-225, B-227, B-228, B-229, B- | |
| 230, B-231, B-232, and B-233. | |

By way of yet further example, Table 5c details additional suitable combinations that may be employed in the methods and compositions of the current invention. The combination may also include an isomer, a pharmaceutically acceptable salt, ester, or prodrug of any of the cyclooxygenase-2 selective inhibitors and/or ACE inhibitors listed in Table 5c.

TABLE 5c

| Cyclooxygenase-2 Selective Inhibitor | ACE inhibitor |
|---|---|
| (S)-6,8-dichloro-2-(trifluoromethyl)-2H-1- | Benazepril |
| benzopyran-3-carboxylic acid | |
| (S)-6,8-dichloro-2-(trifluoromethyl)-2H-1- | Enalapril |
| benzopyran-3-carboxylic acid | |
| (S)-6,8-dichloro-2-(trifluoromethyl)-2H-1- | Fosinopril |
| benzopyran-3-carboxylic acid | |
| (S)-6,8-dichloro-2-(trifluoromethyl)-2H-1- | Lisinopril |
| benzopyran-3-carboxylic acid | |
| (S)-6,8-dichloro-2-(trifluoromethyl)-2H-1- | Moexipril |
| benzopyran-3-carboxylic acid | |
| (S)-6,8-dichloro-2-(trifluoromethyl)-2H-1- | Perindopril |
| benzopyran-3-carboxylic acid | |
| (S)-6,8-dichloro-2-(trifluoromethyl)-2H-1- | Quinapril |
| benzopyran-3-carboxylic acid | |
| (S)-6,8-dichloro-2-(trifluoromethyl)-2H-1- | Ramipril |
| benzopyran-3-carboxylic acid | |
| (S)-6,8-dichloro-2-(trifluoromethyl)-2H-1- | Trandolapril |
| benzopyran-3-carboxylic acid | |
| 6-nitro-2-triflouromethyl-2H-1-benzopyran- | Benazepril |
| 3-carboxylic acid | |
| 6-nitro-2-triflouromethyl-2H-1-benzopyran- | Enalapril |
| 3-carboxylic acid | |
| 6-nitro-2-triflouromethyl-2H-1-benzopyran- | Fosinopril |
| 3-carboxylic acid | |
| 6-nitro-2-triflouromethyl-2H-1-benzopyran- | Lisinopril |
| 3-carboxylic acid | |
| 6-nitro-2-triflouromethyl-2H-1-benzopyran- | Moexipril |
| 3-carboxylic acid | |
| 6-nitro-2-triflouromethyl-2H-1-benzopyran- | Perindopril |
| 3-carboxylic acid | |
| 6-nitro-2-triflouromethyl-2H-1-benzopyran- | Quinapril |
| 3-carboxylic acid | |
| 6-nitro-2-triflouromethyl-2H-1-benzopyran- | Ramipril |
| 3-carboxylic acid | |
| 6-nitro-2-triflouromethyl-2H-1-benzopyran- | Trandolapril |
| 3-carboxylic acid | |
| 6-chloro-8-methyl-2-triflouromethyl-2H-1- | Benazepril |
| benzopyran-3-carboxylic acid | |
| 6-chloro-8-methyl-2-triflouromethyl-2H-1- | Enalapril |
| benzopyran-3-carboxylic acid | |
| 6-chloro-8-methyl-2-triflouromethyl-2H-1- | Fosinopril |
| benzopyran-3-carboxylic acid | |
| 6-chloro-8-methyl-2-triflouromethyl-2H-1- | Lisinopril |
| benzopyran-3-carboxylic acid | |
| 6-chloro-8-methyl-2-triflouromethyl-2H-1- | Moexipril |
| benzopyran-3-carboxylic acid | |
| 6-chloro-8-methyl-2-triflouromethyl-2H-1- | Perindopril |
| benzopyran-3-carboxylic acid | |
| 6-chloro-8-methyl-2-triflouromethyl-2H-1- | Quinapril |
| benzopyran-3-carboxylic acid | |
| 6-chloro-8-methyl-2-triflouromethyl-2H-1- | Ramipril |
| benzopyran-3-carboxylic acid | |
| 6-chloro-8-methyl-2-triflouromethyl-2H-1- | Trandolapril |
| benzopyran-3-carboxylic acid | |
| 6-chloro-2-trifluoromethyl-2H-1- | Benazepril |
| benzopyran-3-carboxylic acid | |
| 6-chloro-2-trifluoromethyl-2H-1- | Enalapril |
| benzopyran-3-carboxylic acid | |
| 6-chloro-2-trifluoromethyl-2H-1- | Fosinopril |
| benzopyran-3-carboxylic acid | |
| 6-chloro-2-trifluoromethyl-2H-1- | Lisinopril |
| benzopyran-3-carboxylic acid | |
| 6-chloro-2-trifluoromethyl-2H-1- | Moexipril |
| benzopyran-3-carboxylic acid | |
| 6-chloro-2-trifluoromethyl-2H-1- | Perindopril |
| benzopyran-3-carboxylic acid | |
| 6-chloro-2-trifluoromethyl-2H-1- | Quinapril |
| benzopyran-3-carboxylic acid | |
| 6-chloro-2-trifluoromethyl-2H-1- | Ramipril |
| benzopyran-3-carboxylic acid | |
| 6-chloro-2-trifluoromethyl-2H-1- | Trandolapril |
| benzopyran-3-carboxylic acid | |
| 6-chloro-7-methyl-2-trifluoromethyl-2H-1- | Benazepril |
| benzopyran-3-carboxylic acid | |
| 6-chloro-7-methyl-2-trifluoromethyl-2H-1- | Enalapril |
| benzopyran-3-carboxylic acid | |
| 6-chloro-7-methyl-2-trifluoromethyl-2H-1- | Fosinopril |
| benzopyran-3-carboxylic acid | |
| 6-chloro-7-methyl-2-trifluoromethyl-2H-1- | Lisinopril |
| benzopyran-3-carboxylic acid | |
| 6-chloro-7-methyl-2-trifluoromethyl-2H-1- | Moexipril |
| benzopyran-3-carboxylic acid | |
| 6-chloro-7-methyl-2-trifluoromethyl-2H-1- | Perindopril |
| benzopyran-3-carboxylic acid | |
| 6-chloro-7-methyl-2-trifluoromethyl-2H-1- | Quinapril |
| benzopyran-3-carboxylic acid | |
| 6-chloro-7-methyl-2-trifluoromethyl-2H-1- | Ramipril |
| benzopyran-3-carboxylic acid | |
| 6-chloro-7-methyl-2-trifluoromethyl-2H-1- | Trandolapril |
| benzopyran-3-carboxylic acid | |
| 8-(1-methylethyl)-2-trifluoromethyl-2H-1- | Benazepril |
| benzopyran-3-carboxylic acid | |
| 8-(1-methylethyl)-2-trifluoromethyl-2H-1- | Enalapril |
| benzopyran-3-carboxylic acid | |
| 8-(1-methylethyl)-2-trifluoromethyl-2H-1- | Fosinopril |
| benzopyran-3-carboxylic acid | |
| 8-(1-methylethyl)-2-trifluoromethyl-2H-1- | Lisinopril |
| benzopyran-3-carboxylic acid | |
| 8-(1-methylethyl)-2-trifluoromethyl-2H-1- | Moexipril |
| benzopyran-3-carboxylic acid | |
| 8-(1-methylethyl)-2-trifluoromethyl-2H-1- | Perindopril |
| benzopyran-3-carboxylic acid | |
| 8-(1-methylethyl)-2-trifluoromethyl-2H-1- | Quinapril |
| benzopyran-3-carboxylic acid | |
| 8-(1-methylethyl)-2-trifluoromethyl-2H-1- | Ramipril |
| benzopyran-3-carboxylic acid | |
| 8-(1-methylethyl)-2-trifluoromethyl-2H-1- | Trandolapril |
| benzopyran-3-carboxylic acid | |
| 6,8-bis(dimethylethyl)-2-trifluoromethyl- | Benazepril |
| 2H-1-benzopyran-3-carboxylic acid | |
| 6,8-bis(dimethylethyl)-2-trifluoromethyl- | Enalapril |
| 2H-1-benzopyran-3-carboxylic acid | |
| 6,8-bis(dimethylethyl)-2-trifluoromethyl- | Fosinopril |
| 2H-1-benzopyran-3-carboxylic acid | |
| 6,8-bis(dimethylethyl)-2-trifluoromethyl- | Lisinopril |
| 2H-1-benzopyran-3-carboxylic acid | |
| 6,8-bis(dimethylethyl)-2-trifluoromethyl- | Moexipril |
| 2H-1-benzopyran-3-carboxylic acid | |
| 6,8-bis(dimethylethyl)-2-trifluoromethyl- | Perindopril |
| 2H-1-benzopyran-3-carboxylic acid | |
| 6,8-bis(dimethylethyl)-2-trifluoromethyl- | Quinapril |
| 2H-1-benzopyran-3-carboxylic acid | |
| 6,8-bis(dimethylethyl)-2-trifluoromethyl- | Ramipril |
| 2H-1-benzopyran-3-carboxylic acid | |
| 6,8-bis(dimethylethyl)-2-trifluoromethyl- | Trandolapril |
| 2H-1-benzopyran-3-carboxylic acid | |
| 7-phenyl-2-trifluoromethyl-2H-1- | Benazepril |
| benzopyran-3-carboxylic acid | |
| 7-phenyl-2-trifluoromethyl-2H-1- | Enalapril |
| benzopyran-3-carboxylic acid | |
| 7-phenyl-2-trifluoromethyl-2H-1- | Fosinopril |
| benzopyran-3-carboxylic acid | |
| 7-phenyl-2-trifluoromethyl-2H-1- | Lisinopril |
| benzopyran-3-carboxylic acid | |
| 7-phenyl-2-trifluoromethyl-2H-1- | Moexipril |
| benzopyran-3-carboxylic acid | |
| 7-phenyl-2-trifluoromethyl-2H-1- | Perindopril |
| benzopyran-3-carboxylic acid | |
| 7-phenyl-2-trifluoromethyl-2H-1- | Quinapril |
| benzopyran-3-carboxylic acid | |
| 7-phenyl-2-trifluoromethyl-2H-1- | Ramipril |
| benzopyran-3-carboxylic acid | |
| 7-phenyl-2-trifluoromethyl-2H-1- | Trandolapril |
| benzopyran-3-carboxylic acid | |
| N-(4-Nitro-2-phenoxy-phenyl)- | Benazepril |
| methanesulfonamide | |
| N-(4-Nitro-2-phenoxy-phenyl)- | Enalapril |
| methanesulfonamide | |
| N-(4-Nitro-2-phenoxy-phenyl)- | Fosinopril |
| methanesulfonamide | |
| N-(4-Nitro-2-phenoxy-phenyl)- | Lisinopril |
| methanesulfonamide | |
| N-(4-Nitro-2-phenoxy-phenyl)- | Moexipril |
| methanesulfonamide | |
| N-(4-Nitro-2-phenoxy-phenyl)- | Perindopril |
| methanesulfonamide | |
| N-(4-Nitro-2-phenoxy-phenyl)- | Quinapril |
| methanesulfonamide | |
| N-(4-Nitro-2-phenoxy-phenyl)- | Ramipril |
| methanesulfonamide | |
| N-(4-Nitro-2-phenoxy-phenyl)- | Trandolapril |
| methanesulfonamide | |
| N-[6-(2,4-difluoro-phenoxy)-1-oxo-indan-5- | Benazepril |
| yl]-methanesulfonamide | |
| N-[6-(2,4-difluoro-phenoxy)-1-oxo-indan-5- | Enalapril |
| yl]-methanesulfonamide | |
| N-[6-(2,4-difluoro-phenoxy)-1-oxo-indan-5- | Fosinopril |
| yl]-methanesulfonamide | |
| N-[6-(2,4-difluoro-phenoxy)-1-oxo-indan-5- | Lisinopril |
| yl]-methanesulfonamide | |
| N-[6-(2,4-difluoro-phenoxy)-1-oxo-indan-5- | Moexipril |
| yl]-methanesulfonamide | |
| N-[6-(2,4-difluoro-phenoxy)-1-oxo-indan-5- | Perindopril |
| yl]-methanesulfonamide | |
| N-[6-(2,4-difluoro-phenoxy)-1-oxo-indan-5- | Quinapril |
| yl]-methanesulfonamide | |
| N-[6-(2,4-difluoro-phenoxy)-1-oxo-indan-5- | Ramipril |
| yl]-methanesulfonamide | |
| N-[6-(2,4-difluoro-phenoxy)-1-oxo-indan-5- | Trandolapril |
| yl]-methanesulfonamide | |
| N-[5-(4-fluoro-phenylsulfanyl)-thiophen-2- | Benazepril |
| yl]-methanesulfonamide | |
| N-[5-(4-fluoro-phenylsulfanyl)-thiophen-2- | Enalapril |
| yl]-methanesulfonamide | |
| N-[5-(4-fluoro-phenylsulfanyl)-thiophen-2- | Fosinopril |
| yl]-methanesulfonamide | |
| N-[5-(4-fluoro-phenylsulfanyl)-thiophen-2- | Lisinopril |
| yl]-methanesulfonamide | |
| N-[5-(4-fluoro-phenylsulfanyl)-thiophen-2- | Moexipril |
| yl]-methanesulfonamide | |
| N-[5-(4-fluoro-phenylsulfanyl)-thiophen-2- | Perindopril |
| yl]-methanesulfonamide | |
| N-[5-(4-fluoro-phenylsulfanyl)-thiophen-2- | Quinapril |
| yl]-methanesulfonamide | |
| N-[5-(4-fluoro-phenylsulfanyl)-thiophen-2- | Ramipril |
| yl]-methanesulfonamide | |
| N-[5-(4-fluoro-phenylsulfanyl)-thiophen-2- | Trandolapril |
| yl]-methanesulfonamide | |
| (5Z)-2-amino-5-[[3,5-bis(1,1- | Benazepril |
| dimethylethyl)-4- | |
| hydroxyphenyl]methylene]-4(5H)- | |
| thiazolone | |
| (5Z)-2-amino-5-[[3,5-bis(1,1- | Enalapril |
| dimethylethyl)-4- | |
| hydroxyphenyl]methylene]-4(5H)- | |
| thiazolone | |
| (5Z)-2-amino-5-[[3,5-bis(1,1- | Fosinopril |
| dimethylethyl)-4- | |
| hydroxyphenyl]methylene]-4(5H)- | |
| thiazolone | |
| (5Z)-2-amino-5-[[3,5-bis(1,1- | Lisinopril |
| dimethylethyl)-4- | |
| hydroxyphenyl]methylene]-4(5H)- | |
| thiazolone | |
| (5Z)-2-amino-5-[[3,5-bis(1,1- | Moexipril |
| dimethylethyl)-4- | |
| hydroxyphenyl]methylene]-4(5H)- | |
| thiazolone | |
| (5Z)-2-amino-5-[[3,5-bis(1,1- | Perindopril |
| dimethylethyl)-4- | |
| hydroxyphenyl]methylene]-4(5H)- | |
| thiazolone | |
| (5Z)-2-amino-5-[[3,5-bis(1,1- | Quinapril |
| dimethylethyl)-4- | |
| hydroxyphenyl]methylene]-4(5H)- | |
| thiazolone | |
| (5Z)-2-amino-5-[[3,5-bis(1,1- | Ramipril |
| dimethylethyl)-4- | |
| hydroxyphenyl]methylene]-4(5H)- | |
| thiazolone | |
| (5Z)-2-amino-5-[[3,5-bis(1,1- | Trandolapril |
| dimethylethyl)-4- | |
| hydroxyphenyl]methylene]-4(5H)- | |
| thiazolone | |
| N-[3-(formylamino)-4-oxo-6-phenoxy-4H- | Benazepril |
| 1-benzopyran-7-yl]-methanesulfonamide | |
| N-[3-(formylamino)-4-oxo-6-phenoxy-4H- | Enalapril |
| 1-benzopyran-7-yl]-methanesulfonamide | |
| N-[3-(formylamino)-4-oxo-6-phenoxy-4H- | Fosinopril |
| 1-benzopyran-7-yl]-methanesulfonamide | |
| N-[3-(formylamino)-4-oxo-6-phenoxy-4H- | Lisinopril |
| 1-benzopyran-7-yl]-methanesulfonamide | |
| N-[3-(formylamino)-4-oxo-6-phenoxy-4H- | Moexipril |
| 1-benzopyran-7-yl]-methanesulfonamide | |
| N-[3-(formylamino)-4-oxo-6-phenoxy-4H- | Perindopril |
| 1-benzopyran-7-yl]-methanesulfonamide | |
| N-[3-(formylamino)-4-oxo-6-phenoxy-4H- | Quinapril |
| 1-benzopyran-7-yl]-methanesulfonamide | |
| N-[3-(formylamino)-4-oxo-6-phenoxy-4H- | Ramipril |
| 1-benzopyran-7-yl]-methanesulfonamide | |
| N-[3-(formylamino)-4-oxo-6-phenoxy-4H- | Trandolapril |
| 1-benzopyran-7-yl]-methanesulfonamide | |
| 6-dioxo-9H-purin-8-yl-cinnamic acid | Benazepril |
| 6-dioxo-9H-purin-8-yl-cinnamic acid | Enalapril |
| 6-dioxo-9H-purin-8-yl-cinnamic acid | Fosinopril |
| 6-dioxo-9H-purin-8-yl-cinnamic acid | Lisinopril |
| 6-dioxo-9H-purin-8-yl-cinnamic acid | Moexipril |
| 6-dioxo-9H-purin-8-yl-cinnamic acid | Perindopril |
| 6-dioxo-9H-purin-8-yl-cinnamic acid | Quinapril |
| 6-dioxo-9H-purin-8-yl-cinnamic acid | Ramipril |
| 6-dioxo-9H-purin-8-yl-cinnamic acid | Trandolapril |
| 8-bromo-5-fluoro-2-trifluoromethyl-2H-1- | Benazepril |
| benzopyran-3-carboxylic acid | |
| 8-bromo-5-fluoro-2-trifluoromethyl-2H-1- | Enalapril |
| benzopyran-3-carboxylic acid | |
| 8-bromo-5-fluoro-2-trifluoromethyl-2H-1- | Fosinopril |
| benzopyran-3-carboxylic acid | |
| 8-bromo-5-fluoro-2-trifluoromethyl-2H-1- | Lisinopril |
| benzopyran-3-carboxylic acid | |
| 8-bromo-5-fluoro-2-trifluoromethyl-2H-1- | Moexipril |
| benzopyran-3-carboxylic acid | |
| 8-bromo-5-fluoro-2-trifluoromethyl-2H-1- | Perindopril |
| benzopyran-3-carboxylic acid | |
| 8-bromo-5-fluoro-2-trifluoromethyl-2H-1- | Quinapril |
| benzopyran-3-carboxylic acid | |
| 8-bromo-5-fluoro-2-trifluoromethyl-2H-1- | Ramipril |
| benzopyran-3-carboxylic acid | |
| 8-bromo-5-fluoro-2-trifluoromethyl-2H-1- | Trandolapril |
| benzopyran-3-carboxylic acid | |

### Diagnosis of a Vaso-Occlusion

One aspect of the invention encompasses diagnosing a subject in need of treatment or prevention for a vaso-occlusive event. A number of suitable methods for diagnosing a vaso-occlusion may be used in the practice of the invention. In one such method, ultrasound may be employed. This method examines the blood flow in the major arteries and veins in the arms and legs with the use of ultrasound (high-frequency sound waves). In one embodiment, the test may combine Doppler® ultrasonography, which uses audio measurements to “hear” and measure the blood flow and duplex ultrasonography, which provides a visual image. In an alternative embodiment, the test may utilize multifrequency ultrasound or multifrequency transcranial Doppler® (MTCD) ultrasound.

Another method that may be employed encompasses injection of the subject with a compound that can be imaged. In one alternative of this embodiment, a small amount of radioactive material is injected into the subject and then standard techniques that rely on monitoring blood flow to detect a blockage, such as magnetic resonance direct thrombus imaging (MRDTI), may be utilized to image the vaso-occlusion. In an alternative embodiment, ThromboView® (commercially available from Agenix Limited) uses a clot-binding monoclonal antibody attached to a radiolabel. In addition to the methods identified herein, a number of other suitable methods known in the art for diagnosis of vaso-occlusive events may be utilized.

### Indications to be Treated

Generally speaking, the composition comprising a therapeutically effective amount of a chromene or phenyl acetic acid cyclooxygenase-2 selective inhibitor or an isomer, ester, a pharmaceutically acceptable salt or a prodrug thereof, and a therapeutically effective amount of an ACE inhibitor or a pharmaceutically acceptable salt or prodrug thereof may be employed to treat a number of conditions resulting from a reduction in blood flow to the central nervous system.

In some aspects, the invention provides a method to treat a central nervous system cell to prevent damage in response to a decrease in blood flow to the cell. Typically the severity of damage that may be prevented will depend in large part on the degree of reduction in blood flow to the cell and the duration of the reduction. By way of example, the normal amount of perfusion to brain gray matter in humans is about 60 to 70 mL/100 g of brain tissue/min. Death of central nervous system cells typically occurs when the flow of blood falls below approximately 8-10 mL/100 g of brain tissue/min, while at slightly higher levels (i.e. 20-35 mL/100 g of brain tissue/min) the tissue remains alive but not able to function. In one embodiment, apoptotic or necrotic cell death may be prevented. In still a further embodiment, ischemic-mediated damage, such as cytoxic edema or central nervous system tissue anoxemia, may be prevented. In each embodiment, the central nervous system cell may be a spinal cell or a brain cell.

Another aspect of the invention encompasses administrating the composition to a subject to treat a central nervous system ischemic condition. A number of central nervous system ischemic conditions may be treated by the composition of the invention. In one embodiment, the ischemic condition is a stroke that results in any type of ischemic central nervous system damage, such as apoptotic or necrotic cell death, cytoxic edema or central nervous system tissue anoxemia. The stroke may impact any area of the brain or be caused by any etiology commonly known to result in the occurrence of a stroke. In one alternative of this embodiment, the stroke is a brain stem stroke. Generally speaking, brain stem strokes strike the brain stem, which control involuntary life-support functions such as breathing, blood pressure, and heartbeat. In another alternative of this embodiment, the stroke is a cerebellar stroke. Typically, cerebellar strokes impact the cerebellum area of the brain, which controls balance and coordination. In still another embodiment, the stroke is an embolic stroke. In general terms, embolic strokes may impact any region of the brain and typically result from the blockage of an artery by a vaso-occlusion. In yet another alternative, the stroke may be a hemorrhagic stroke. Like embolic strokes, hemorrhagic stroke may impact any region of the brain, and typically result from a ruptured blood vessel characterized by a hemorrhage (bleeding) within or surrounding the brain. In a further embodiment, the stroke is a thrombotic stroke. Typically, thrombotic strokes result from the blockage of a blood vessel by accumulated deposits.

In another embodiment, the ischemic condition may result from a disorder that occurs in a part of the subject's body outside of the central nervous system, but yet still causes a reduction in blood flow to the central nervous system. These disorders may include, but are not limited to a peripheral vascular disorder, a venous thrombosis, a pulmonary embolus, a myocardial infarction, a transient ischemic attack, unstable angina, or sickle cell anemia. Moreover, the central nervous system ischemic condition may occur as result of the subject undergoing a surgical procedure. By way of example, the subject may be undergoing heart surgery, lung surgery, spinal surgery, brain surgery, vascular surgery, abdominal surgery, or organ transplantation surgery. The organ transplantation surgery may include heart, lung, pancreas or liver transplantation surgery. Moreover, the central nervous system ischemic condition may occur as a result of a trauma or injury to a part of the subject's body outside the central nervous system. By way of example the trauma or injury may cause a degree of bleeding that significantly reduces the total volume of blood in the subject's body. Because of this reduced total volume, the amount of blood flow to the central nervous system is concomitantly reduced. By way of further example, the trauma or injury may also result in the formation of a vaso-occlusion that restricts blood flow to the central nervous system.

Of course it is contemplated that the composition may be employed to treat a number of central nervous system ischemic conditions irrespective of the cause of the particular condition. In one embodiment, the ischemic condition results from a vaso-occlusion. The vaso-occlusion may be any type of occlusion, but is typically a cerebral thrombosis or a cerebral embolism. In a further embodiment, the ischemic condition may result from a hemorrhage. The hemorrhage may be any type of hemorrhage, but is generally a cerebral hemorrhage or a subarachnoid hemorrhage. In still another embodiment, the ischemic condition may result from the narrowing of a vessel. Generally speaking, the vessel may narrow as a result of a vasoconstriction such as occurs during vasospasms, or due to arteriosclerosis. In yet another embodiment, the ischemic condition results from an injury to the brain or spinal cord.

In yet another aspect, the composition is administered to reduce infarct size of the ischemic core following a central nervous system ischemic condition. Moreover, the composition may also be beneficially administered to reduce the size of the ischemic penumbra or transitional zone following a central nervous system ischemic condition

In addition to a chromene or phenyl acetic acid cyclooxygenase-2 selective inhibitor and an ACE inhibitor, the composition of the invention may also include a number of other agents that ameliorates the effect of a reduction in blood flow to the central nervous system. In one embodiment, the agent is an anticoagulant including thrombin inhibitors such as heparin and Factor Xa inhibitors such as warafin. In another embodiment, the agent is a thrombolytic agent including tissue plasminogen activator, urokinase, and desmoteplase (vampire bat plasminogen activator). In an additional embodiment, the agent is an anti-platelet inhibitor such as a GP IIb/IIIa inhibitor. Additional agents include but are not limited to, HMG-COA synthase inhibitors; squalene epoxidase inhibitors; squalene synthetase inhibitors (also known as squalene synthase inhibitors); acyl-coenzyme A; cholesterol acyltransferase (ACAT) inhibitors; probucol; niacin; fibrates such as clofibrate, fenofibrate, and gemfibrizol; cholesterol absorption inhibitors; bile acid sequestrants; LDL (low density lipoprotein) receptor inducers; vitamin B₆ (also known as pyridoxine) and the pharmaceutically acceptable salts thereof such as the HCl salt; vitamin B₁₂ (also known as cyanocobalamin); β-adrenergic receptor blockers; folic acid or a pharmaceutically acceptable salt or ester thereof such as the sodium salt and the methylglucamine salt; and anti-oxidant vitamins such as vitamin C and E and beta carotene.

In a further aspect, the composition may be employed to reverse or lessen central nervous system cell damage following a traumatic brain or spinal cord injury. Traumatic brain or spinal cord injury may result from a wide variety of causes including, for example, blows to the head or back from objects; penetrating injuries from missiles, bullets, and shrapnel; falls; skull fractures with resulting penetration by bone pieces; and sudden acceleration or deceleration injuries. The composition of the invention may be beneficially utilized to treat the traumatic injury irrespective of its cause.

The composition may also beneficially be employed to increase recovery of neural cell function following brain or spinal cord injury. Generally speaking, when neurons are lost due to disease or trauma, they are not replaced. Rather, the remaining neurons must adapt to whatever loss occurred by altering their function or functional relationship relative to other neurons. Following injury, neural tissue begins to produce trophic repair factors, such as nerve growth factor and neuron cell adhesion molecules, which retard further degeneration and promote synaptic maintenance and the development of new synaptic connections. But, as the lost cells are not replaced, existing cells must take over some of the functions of the missing cells, i.e., they must “learn” to do something new. In part, recovery of function from brain traumatic damage involves plastic changes that occur in brain structures other than those damaged. Indeed, in many cases, recovery from brain damage represents the taking over by healthy brain regions of the functions of the damaged area. Thus the composition of the present invention may be administered to facilitate learning of new functions by uninjured brain areas to compensate for the loss of function by other regions.

### EXAMPLES

The following examples are intended to provide illustrations of the application of the present invention. The following examples are not intended to completely define or otherwise limit the scope of the invention.

In the examples below, a combination therapy contains an ACE inhibitor and a chromene or phenyl acetic acid COX-2 selective inhibitor. The efficacy of such combination therapy can be evaluated in comparison to a control treatment such as a placebo treatment, administration of a chromene or phenyl acetic acid COX-2 inhibitor only, or administration of an ACE inhibitor only. By way of example, a combination therapy may contain benazepril and B-3, enalapril and B-3, moexipril and B-27, or ramipril and B-131. It should be noted that these are only several examples, and that any of the ACE inhibitors and chromene or phenyl acetic acid COX-2 inhibitors of the present invention may be tested as a combination therapy. The dosages of an ACE inhibitor and chromene or phenyl acetic acid COX-2 inhibitor in a particular therapeutic combination may be readily determined by a skilled artisan conducting the study. The length of the study treatment will vary on a particular study and can also be determined by one of ordinary skill in the art. By way of example, the combination therapy may be administered for 12 weeks. The ACE inhibitor and chromene or phenyl acetic acid COX-2 inhibitor can be administered by any route as described herein, but are preferably administered orally for human subjects.

### Example 1

### Evaluation of COX-1 and COX-2 Activity in vitro

The COX-2 inhibitors suitable for use in this invention exhibit selective inhibition of COX-1 over COX-2, as measured by IC₅₀ values when tested in vitro according to the following activity assays.

### Preparation of Recombinant COX Baculoviruses

Recombinant COX-1 and COX-2 are prepared as described by Gierse et al, [J. Biochem., 305, 479-84 (1995)]. A 2.0 kb fragment containing the coding region of either human or murine COX-1 or human or murine COX-2 is cloned into a BamH1 site of the baculovirus transfer vector pVL1393 (Invitrogen) to generate the baculovirus transfer vectors for COX-1 and COX-2 in a manner similar to the method of D. R. O'Reilly et al (Baculovirus Expression Vectors: A Laboratory Manual (1992)). Recombinant baculoviruses are isolated by transfecting 4 μg of baculovirus transfer vector DNA into SF9 insect cells (2×10⁸) along with 200 μg of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses are purified by three rounds of plaque purification and high titer (10⁷-10⁸ pfu/mL) stocks of virus are prepared. For large scale production, SF9 insect cells are infected in 10 liter fermentors (0.5×10⁶/mL) with the recombinant baculovirus stock such that the multiplicity of infection is 0.1. After 72 hours the cells are centrifuged and the cell pellet is homogenized in Tris/Sucrose (50 mM: 25%, pH 8.0) containing 1% 3-[(3-cholamidopropyl)-dimethylammonio]-1-propanesulfonate (CHAPS). The homogenate is centrifuged at 10,000×G for 30 minutes, and the resultant supernatant is stored at −80° C. before being assayed for COX activity.

### Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidonic acid (10 μM). Compounds are pre-incubated with the enzyme for 10-20 minutes prior to the addition of arachidonic acid. Any reaction between the arachidonic acid and the enzyme is stopped after ten minutes at 37° C. by transferring 40 μl of reaction mix into 160 μl ELISA buffer and 25 μM indomethacin. The PGE2 formed is measured by standard ELISA technology (Cayman Chemical).

### Fast Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (0.05 M Potassium phosphate, pH 7.5, 2 μM phenol, 1 μM heme, 300 μM epinephrine) with the addition of 20 μl of 100 μM arachidonic acid (10 μM). Compounds are pre-incubated with the enzyme for 10 minutes at 25° C. prior to the addition of arachidonic acid. Any reaction between the arachidonic acid and the enzyme is stopped after two minutes at 37° C. by transferring 40 μl of reaction mix into 160 μl ELISA buffer and 25 μM indomethacin. Indomethacin, a non-selective COX-2/COX-1 inhibitor, may be utilized as a positive control. The PGE₂ formed is typically measured by standard ELISA technology utilizing a PGE2 specific antibody, available from a number of commercial sources.

Each compound to be tested may be individually dissolved in 2 ml of dimethyl sulfoxide (DMSO) for bioassay testing to determine the COX-1 and COX-2 inhibitory effects of each particular compound. Potency is typically expressed by the IC₅₀ value expressed as g compound/ml solvent resulting in a 50% inhibition of PGE2 production. Selective inhibition of COX-2 may be determined by the IC₅₀ ratio of COX-1/COX-2.

By way of example, a primary screen may be performed in order to determine particular compounds that inhibit COX-2 at a concentration of 10 μg/ml. The compound may then be subjected to a confirmation assay to determine the extent of COX-2 inhibition at three different concentrations (e.g., 10 ug/ml, 3.3 ug/ml and 1.1 ug/ml). After this screen, compounds can then be tested for their ability to inhibit COX-1 at a concentration of 10 ug/ml. With this assay, the percentage of COX inhibition compared to control can be determined, with a higher percentage indicating a greater degree of COX inhibition. In addition, the IC₅₀ value for COX-1 and COX-2 can also be determined for the tested compound. The selectivity for each compound may then be determined by the IC₅₀ ratio of COX-1/COX-2, as set-forth above.

### Example 2

The laboratory animal study can generally be performed as described in Tanaka et al., Neurochemical Research, Vol. 20, No. 6, 1995, pp. 663-667.

Briefly, the study can be performed with about 30 gerbils, with body weights of 65 to 80 grams. The animals are anesthetized with ketamine (100mg/kg body weight, i.p.), and silk threads are placed around both common carotid arteries without interrupting carotid artery blood flow. On the next day, bilateral common carotid arteries are exposed and then occluded with surgical clips after light ether anesthesia (see, e.g., Ogawa et al., Adv. Exp. Med. Biol., 287:343-347, and Ogawa et al., Brain Res., 591:171-175). Carotid artery blood flow is restored by releasing the clips after 5 minutes of occlusion. Body temperature is maintained about 37° C. using a heating pad and an incandescent lamp. Control animals are operated on in a similar manner but the carotid arteries are not occluded. The combination therapy is administered immediately and 6 and 12 hours after recirculation in the ischemia group, whereas sham-operated animals receive placebo, which may be, e.g., the vehicle used to administer the combination therapy. Gerbils are sacrificed by decapitation 14 days after recirculation. The brain is removed rapidly and placed on crushed dry-ice to freeze the tissue.

The brain tissue can then be examined histologically for the effects of combination therapy in comparison to the placebo. For example, each brain is cut into 14 μm thick sections at −15° C. Coronal sections that include the cerebral cortex and hippocampal formation are thawed, mounted onto gelatin-coated slides, dried completely, and fixed with 10% formalin for 2 hours. The sections are stained with hematoxylin-eosin and antibodies to glial fibrillary acidic protein (GFAP), which can be commercially obtained from, e.g., Nichirei, Tokyo, Japan. Immune complexes are detected by the avidin-biotin interaction and visualized with 3,3′-diaminobenzidine tetrahydrochloride. Sections that are used as controls are stained in a similar manner without adding anti-GFAP antibody. The densities of living pyramidal cells and GFAP-positive astrocytes in the typical CA1 subfield of the hippocampus are calculated by counting the cells and measuring the total length of the CA1 cell layer in each section from 250× photomicrographs. The average densities of pyramidal cells and GFAP-positive astrocytes in the CA1 subfield for each gerbil are obtained from counting cells in one unit area in each of these sections of both left and right hemispheres.

The effects of the combination therapy in comparison with the placebo can be determined both qualitatively and quantitatively. For example, the appearance of CA1 pyramidal neurons and pyramidal cell density in the CA1 subfield may be used to assess the efficacy of the treatment. In addition, immunohistological analysis can reveal the efficacy of combination by evaluating the presence or absence of hypertrophic GFAP-positive astrocytes in the CA1 region of treated gerbils, since the sham-operated animals should have few GFAP-positive astrocytes.

### Example 3

Rat middle cerebral artery occlusion (MCAO) models are well known in the art and useful in assessing a neuroprotective drug efficacy in stroke. By way of example, the methods and materials for MCAO model described in Turski et al. (Proc. Natl. Acad, Sci. USA, Vol. 95, pp.10960-10965, September 1998) may be modified for testing the combination therapy as described above for cerebral ischemia treatment.

The permanent middle cerebral artery occlusion can be established by means of microbipolar permanent coagulation in, e.g., Fisher 344 rats (260-290 grams) anesthetized with halothane as described previously in, e.g., Lippert et al., Eur. J. Pharmacol., 253, pp.207-213, 1994. To determine the efficacy of the combination treatment and the therapeutic window for such treatment, the combination therapy can be administered, e.g., intravenously over 6 hours beginning 1, 2,4, 5, 6, 7, 12, or 24 hours after MCAO. It should be noted that different doses, routes of administrations, and times of administration can also be readily tested. Furthermore, the experiment should be controlled appropriately, e.g. by administering placebo to a set of MCAO-induced rats. To evaluate the efficacy of the combination therapy, the size of infarct in the brain can be estimated stereologically, e.g., seven days after MCAO, by means of advanced image analysis.

In addition, the assessment of neuroprotective action against focal cerebral reperfusion ischemia can be performed in Wistar rats (250-300 grams) that are anesthetized with halothane and subjected to temporary occlusion of the common carotid arteries and the right middle cerebral artery (CCA/MCAO) for 90 minutes. CCAs can be occluded by means of silastic threads placed around the vessels, and MCA can be occluded by means of a steel hook attached to a micromanipulator. Blood flow stop can be verified by microscopic examination of the MCA or laser doppler flowmetry. Different doses of combination therapy can then be administered over, e.g., 6 hours starting immediately after the beginning of reperfusion or, e.g., 2 hours after the onset of reperfusion. As mentioned previously, the size of infarct in the brain can be estimated, for example, stereologically seven days after CCA/MCAO by means of image analysis.

### Example 4

The following procedures can be performed as described in, e.g., Nogawa et al., Journal of Neuroscience, 17(8):2746-2755, Apr. 15, 1997.

The middle cerebral artery (MCA) is transiently occluded in a number of Sprague Dawley rats, weighing 275-310 grams, using an intravascular occlusion model, as described in, e.g., Longa et al., Stroke 20:84-91, 1989, ladecola et al., Stroke 27:1373-1380, 1996,and Zhang etal., Stroke 27:317-323. A skilled artisan can readily determine the appropriate number of animals to be used for a particular experiment. Under halothane anesthesia (induction 5%, maintenance 1%), a 4-0 nylon monofilament with a rounded tip is inserted centripetally into the external carotid artery and advanced into the internal carotid artery until it reaches the circle of Willis. Throughout the procedure, body temperature is maintained at 37°±0.5° C. by a thermostatically controlled lamp. Two hours after induction of ischemia, rats are reanesthetized, and the filament is withdrawn, as described in, e.g., Zhang et al., Stroke 27:317-323. Animals are then returned to their cages and closely monitored until recovery from anesthesia.

Under halothane anesthesia, the femoral artery is cannulated, and rats are placed on a stereotaxic frame. The arterial catheter is used for monitoring of arterial pressure and other parameters at different times after MCA occlusion. The MCA is occluded for 2 hours, as described above, and treatments are started, e.g., 6 hours after induction of ischemia. In one group of rats (e.g., 6), the combination therapy is administered, e.g., intraperitoneally, twice a day for 3 days. It should be noted that different doses, routes of administration, and times of administration can also be readily tested. A second group of rats is treated with a placebo administered in the same manner. Arterial pressure, rectal temperature, and plasma glucose are measured three times a day during the experiment. Arterial hematocrit and blood gases are measured before injection and 24, 48, and 72 hours after ischemia. Three days after MCA occlusion, brains are removed and frozen in cooled isopentane (−30° C.). Coronal forebrain sections (30 μM thick) are serially cut in cryostat, collected at 300 μm intervals, and stained with thionin for determination of infarct volume by an image analyzer (e.g., MCID, Imaging Research), as described in ladecola et al., J Cereb Blood Flow Metab, 15:378-384,1995. Infarct volume in cerebral cortex is corrected for swelling according to the method of Lin et al., Stroke 24:117-121, 1993, which is based on comparing the volumes of neocortex ipsilateral and contralateral to the stroke. The correction for swelling is needed to factor out the contribution of ischemic swelling to the total volume of the lesion (see Zhang and ladecola, J Cereb Blood Flow Metab, 14:574-580, 1994). Reduction of infarct size in combination therapy-treated animals compared to animals receiving placebo is indicative of the efficacy of the combination therapy.

It should be noted that all of the above-mentioned procedures could be modified for a particular study, depending on factors such as a drug combination used, length of the study, subjects that are selected, etc. Such modifications can be designed by a skilled artisan without undue experimentation.

1. A method for treating a stroke, the method comprising:
(a) diagnosing a subject in need of treatment for a stroke; and
(b) administering to the subject a chromene or phenyl acetic acid cyclooxygenase-2 selective inhibitor or an isomer, a pharmaceutically acceptable salt, ester, or prodrug thereof and an ACE inhibitor or an isomer, a pharmaceutically acceptable salt, ester, or prodrug thereof.

2. The method of claim 1 wherein the cyclooxygenase-2 selective inhibitor has a selectivity ratio of COX-1 IC₅₀ to COX-2 IC₅₀ not less than about 50.

3. The method of claim 1 wherein the cyclooxygenase-2 selective inhibitor has a selectivity ratio of COX-1 IC₅₀ to COX-2 IC₅₀ not less than about 100.

4. The method of claim 1 wherein the cyclooxygenase-2 selective inhibitor is a chromene compound.

5. The method of claim 4 wherein the chromene compound is a benzopyran or substituted benzopyran analog.

6. The method of claim 1 wherein the cyclooxygenase-2 selective inhibitor is selected from the group consisting of (S)-6,8-dichloro-2-(trifluoromethyl)-2H-1-benzopyran-3-carboxylic acid, 6-nitro-2-triflouromethyl-2H-1-benzopyran-3-carboxylic acid, 6-chloro-8-methyl-2-triflouromethyl-2H-1-benzopyran-3-carboxylic acid, 6-chloro-2-trifluoromethyl-2H-1-benzopyran-3-carboxylic acid, 6-chloro-7-methyl-2-trifluoromethyl-2H-1-benzopyran-3-carboxylic acid, 8-(1-methylethyl)-2-trifluoromethyl-2H-1-benzopyran-3-carboxylic acid, 6,8-bis(dimethylethyl)-2-trifluoromethyl-2H-1-benzopyran-3-carboxylic acid, 7-phenyl-2-trifluoromethyl-2H-1-benzopyran-3-carboxylic acid, 8-bromo-5-fluoro-2-trifluoromethyl-2H-1-benzopyran-3-carboxylic acid, N-(4-Nitro-2-phenoxy-phenyl)-methanesulfonamide, N-[6-(2,4-difluoro-phenoxy)-1-oxo-indan-5-yl]-methanesulfonamide, N-[5-(4-fluoro-phenylsulfanyl)-thiophen-2-yl]-methanesulfonamide, (5Z)-2-amino-5-[[3,5-bis(1,1-dimethylethyl)-4-hydroxyphenyl]methylene]-4(5H)-thiazolone, N-[3-(formylamino)-4-oxo-6-phenoxy-4H-1-benzopyran-7-yl]-methanesulfonamide, and 6-dioxo-9H-purin-8-yl-cinnamic acid, or is an isomer, a pharmaceutically acceptable salt, ester, or prodrug thereof.

7. The method of claim 6 wherein the cyclooxygenase-2 selective inhibitor is (S)-6,8-dichloro-2-(trifluoromethyl)-2H-1-benzopyran-3-carboxylic acid, or an isomer, a pharmaceutically acceptable salt, ester, or prodrug thereof.

8. The method of claim 1 wherein the ACE inhibitor is selected from the group consisting of:
enalapril;
captopril;
benazepril;
fosinopril;
lisinopril;
moexipril;
perindopril;
quinapril;
ramipril;
trandolapril;
1-[3-(benzoylthio)-2-methyl-1-oxopropyl]-4-(phenylthio)-,[1(R*),2alpha,4alpha]-L-proline;
2-[2-[[1-(ethoxycarbonyl)-3-phenylpropyl]amino]-1-oxopropyl]-,[3S-[2[R*(R*)],3R*]]-2-azabicyclo[2.2.2]octane-3-carboxylic acid;
N-[2(S)-benzyl-3-sulfanylpropionyl]-4-(2-thiazolyl)-L-phenylalanine;
2S-[1[R*(R*)],2R*]]-1-[2-[[1-(ethoxycarbonyl)-3-phenylpropyl]amino]-1-oxopropyl]-2,3-dihydro-1H-indole-2-carboxylic acid monosodium salt;
5-(1,1-dimethylethyl)-3-[2-[[1-(ethoxycarbonyl)-3-phenylpropyl]amino]-1-oxopropyl]-2,3-dihydro-,[2S[2R*,3[R*(R*)]]]-1,3,4-thiadiazole-2-carboxylic acid;
N-(N2-acetyl-L-lysyl)-L-2-[[1-[[[1-carboxy-2-(4-hydroxyphenyl)ethyl]amino]carbonyl]cyclopentyl]methyl]-b-alanine;
N-[[1-(2-carboxy-4-phenylbutyl)cyclopentyl]carbonyl]-L-tryptophan;
1-(N-((1S)-1-carboxy-3-phenylpropyl)-(S)-alanyl-4-(N-methyl-N-(6-chloro-7-sulfamoyl)-3,4-dihydro-1,2,4-benzothiadiazine-1,1-dioxide-3-yl)methyl)aminoproline;
6-[[1-(ethoxycarbonyl)-3-phenylpropyl]amino]tetrahydro-5-oxo-2-(2-thienyl)-,[2S-[2alpha,6beta(R*)]]-1,4-thiazepine-4(5H)-acetic acid;
1-[2-[[1-(ethoxycarbonyl)-3-phenylpropyl]amino]-1-oxopropyl]octahydro-,[2S-[1[R*(R*)],2alpha,3aalpha,7abeta]]-1H-Indole-2-carboxylic acid;
1-[N-(8-amino-1-carboxyoctyl)-L-alanyl]-,(S)-L-proline;
N-[[1-[[2-(acetylthio)-3-methyl-1-oxobutyl]amino]cyclopentyl]carbonyl]-ethyl ester (S)- L-tryptophan;
1-[(2S)-3-(acetylthio)-2-methyl-1-oxopropyl]-L-prolyl-L-phenylalanine;
3-[[(1S)-1-(ethoxycarbonyl)-3-phenylpropyl]amino]-2,3,4,5-tetrahydro-2-oxo-(3S)-1H-1-benzazepine-1-acetic acid;
[S-(R*,R*)]-2,3,4,5-tetrahydro-3-[(2-mercapto-1-oxo-3-phenylpropyl)amino]-2-oxo-1H-benzazepine-1-acetic acid;
N-[4-(2,3-dihydro-2-benzofuranyl)-1-(ethoxycarbonyl)butyl]-L-alanyl-L-proline;
N-[(S)-1-carboxy-5-(4-[(S)-2-hydroxy-3-isopropylaminoproproxy]-1H-indole-2-carboxamido)pentyl]-(S)-alanyl-(S)-proline;
1-[(2S)-3-mercapto-2-methyl-1-oxopropyl]-L-proline;
1-[6-amino-2-[[hydroxy(4-phenylbutyl)phosphinyl]oxy]-1-oxohexyl]-,(S)-L-proline;
(3S ,6S)-4-oxo-3-(sulfanylmethyl)-5-azabicyclo[10.3.1]hexadeca-1 (16),12,14-triene-6-carboxylic acid;
2,3,4,5,6,7,8,9-octahydro-2-(mercaptomethyl)-3-oxo-,(2S,5S)-1H-4-benzazacycloundecine-5-carboxylic acid;
10-[(3-phenyl-2-mercapto-1-oxo)propylamino]-9-oxo-8-azatricyclo[4.3.2]-1,3,5-hexahydrododecan-7-carboxylic acid;
N-[1-[2(S)-(acetylsulfanyl)-3-methylbutyramido]cyclopent-1-ylcarbonyl]-4-O-methyl-L-tyrosine ethyl ester;
9-[[(1S)-1-(ethoxycarbonyl)-3-phenylpropyl]amino]octahydro-10-oxo-(1S,9S)-6H-pyridazino[1,2-a][1,2]diazepine-1-carboxylic acid;
3-[[1-carboxy-5-(4-piperidinyl)pentyl]amino]3,4,dihydro-4-oxo,[s-(R*,S*)]-1,5-benzothiazepine-5(2H)-acetic acid;
N-(2,3-dihydro-1H-inden-2-yl)-N-[N-[1-(ethoxycarbonyl)-3-phenylpropyl]-L-alanyl]-(S)-glycine;
(2S,3aS,7aS)-1-[N-(3-pyridylcarbonyl)-1-lysyl-gamma-dglutamyl]octahydroindole-2-carboxylic acid;
N-[(1S)-1-(ethoxycarbonyl)-3-phenylpropyl]-L-alanyl-L-proline;
N-[(1S)-1-carboxy-3-phenylpropyl]-L-alanyl- L-proline;
(S)-N-[N-[1-[ethoxycarbonyl]-3-phenylpropyl]-L-alanyl]-N-[2-ethoxyethoxy]-glycine;
(3R,6S ,9R,9aR)-6-[(2S,3S)-2-acetylthio-3-methylpentylamido]-9-methyl-5-oxooctahydroazepino[2,1-b]thiazole-3-carboxylic acid;
1,2,3,4,6,7,8,12b-octahydro-7-[(2-mercapto-3-methyl-1-oxopentyl)amino]-6-oxo-11-phenyl-[4S-[4a,7a(2R*,3R*),12bb]]-pyrido[2,1-a][2]benzazepine-4-carboxylic acid;
octahydro-6-[((2-mercapto-3-methyl-1-oxopentyl)amino)]-5-oxo-,[(3R-[(3alpha,6alpha(2S*,3S*),9alphabeta)])]-thiazolo[(3,2-a)]azepine-3-carboxylic acid;
(−)-(S)-N-[2-acetylthiomethyl)-3-(3,4-methylenedioxyphenyl)propanoyl]-(S)-alanine benzyl ester;
N2-acetyl-N5-formyl-N5-hydroxy-L-ornithyl-N-[3-[(2S,5R)-5-[3-(formylhydroxyamino)propyl]-3,6-dioxo-2-piperazinyl]propyl]-N-hydroxy-L-serinamide;
4-cyclohexyl-1-[[[2-methyl-1-(1-oxopropoxy)propoxy](4-phenylbutyl)phosphinyl]acetyl]-,[1[S*(R*)],2alpha,4beta]-,sodium salt L-proline;
[4(S)-cyclohexyl-2(R)-I-prolin-1yl]carbonylmethyl-(4-phenylbutyl)phosphinic acid;
2(R)-[3-mercapto-2(S)-methylpropranoyloxy]-3-(methylthio)propanoic acid;
hexahydro-6-[[[2S]-2-mercapto-1-oxo-3-phenylpropyl]amino]-2,2-dimethyl-7-oxo-,[6S]-1H-1-acetic acid;
N-[3-(acetylthio)-2-(1,3-benzodioxol-5-ylmethyl)-1-oxopropyl]-,phenylmethyl ester,(S)-glycine;
2-[[[2-(hydroxyamino)-2-oxoethyl]-N-methylamino]carbonyl]-,(+)-cis-(1S,2R)-cyclohexanecarboxylic acid;
3-[2-[[1-(ethoxycarbonyl)-3-phenylpropyl]amino]-1-oxopropyl]-1-methyl-2-oxo-,monohydrochloride[4S-[3[R*(R*)],4R*]]-4-imidazolidinecarboxylic acid;
3-[(5-amino-1-carboxypentyl)amino]-2,3,4,5-tetrahydro-2-oxo-,dihydrobromide,[S-(R*,R*)]-1H-1-benzazepine-1-acetic acid;
N2-[(1S)-1-carboxy-3-phenylpropyl]-L-lysyl-,dihydrate L-proline;
7-[(1-carboxy-3-phenylpropyl)amino]-1,2,3,4,6,7,8,12a-octahydro-6-oxo-,[4S-[4alpha,7alpha,(R*),12bbeta]]-pyrido[2,1-a][2]benzazepine-4-carboxylic acid;
4(S)-4alpha,7alpha(R*),12bbeta]-7-[1-(ethoxycarbonyl)-3-phenylpropylamino]-1,2,3,4,5,6,7,8,12b-octahydro-6-oxopyrido[2,1-a][2]benzazepine-4-carboxylic acid diphenylmethyl ester;
(4S,7S,12bR)-7-[2(S)-(acetylthio)-3-phenylpropionamido]-6-oxo-1,2,3,4,6,7,8,12b-octahydropyrido[2,1-a][2]benzazepine-4-carboxylic acid;
6-oxo-7(S)-[3-phenyl-2(R)-sulfanylpropanamido]-6-oxo-1,2,3,4,6,7,8,12b(R)-octahydropyrido[2,1-a]-2-benzazepine-4(S)-carboxylic acid;
7-[[[2-(carboxymethyl)-2,3-dihydro-1H-inden-2-yl]carbonyl]amino]-1,2,3,4,6,7,8,12b-octahydro-6-oxo-,1(4S,7S,12bR)-Pyrido[2,1-a][2]benzazepine-4-carboxylic acid;
N-[(2S,3R)-2-benzoylthiomethyl-1-oxo-3-phenylbutyl]-(S)-alanine;
2-[2-[[1-(ethoxycarbonyl )-3-phenylpropyl]amino]-1-oxopropyl]-1,2,3,4-tetrahydro-6,7-dimethoxy-,[3S-[2[R*(R*)],3R*]]-monohydrochloride-3-isoquinolinecarboxylic acid;
2-[(2S)-2-[[(1S)-1-carboxy-3-phenylpropyl]amino]-1-oxopropyl]-1,2,3,4-tetrahydro-6,7-dimethoxy-,(3S)-3-isoquinolinecarboxylic acid;
1-[3-[[2-[(cyclohexylcarbonyl)amino]-1′-oxopropyl]thio]-2-methyl-1-oxopropyl]-,calcium salt (2:1) [R-(R*,S*)]-L-proline;
1-[2-methyl-3-(nitrosothio)-1-oxopropyl]-,(S)-L-proline;
(4S,7S,10aS)-octahydro-4-[(S)-alpha-mercaptohydrocinnamido]-5-oxo-7H-pyrido[2,1-b][1,3]thiazepine-7-carboxylic acid;
1-[(2S)-2-[[(1S)-1-(ethoxycarbonyl)butyl]amino]-1-oxopropyl]octahydro-,(2S,3aS,7aS)-1H-indole-2-carboxylic acid;
(2S,3aS,7aS)-1-[(S)-N-[(S)-1-carboxybutyl]alanyl]hexahydro-2-indolinecarboxylic acid;
2-[2[(-1-carboxy-3-phenylpropyl)amino-1-oxopropyl]-1,2,3,4-tetrahydro-,[3S-[2[R*(R*)],3R*]]-3-isoquinolinecarboxylic acid;
1-[2-[[1-(ethoxycarbonyl)-3-phenylpropyl]amino]-1-oxopropyl]octahydro-,[2S-[1[R*(R*)],2alpha,3abeta,6abeta]]-cyclopenta[b]pyrrole-2-carboxylic acid;
N-[2(R)-mercapto-1-oxo-3-(4-oxyphenyl)propyl-glycyl]-[4(S)-(3-oxyphenyl)]-proline;
2-(2-hydroxyphenyl)-3-(3-mercapto-1-oxopropyl)-,(2R,4R)-4-thiazolidinecarboxylic acid;
N-acetyl-L-a-aspartyl-L-phenylalanyl-y(PO(OH)—CH2)-L-alanyl-L-alaninamide;
2(S)-[3-[2(S)-carboxy-2-hydroxyethyl]-3-isobutylureido]-3-(2-naphthyl)propionic acid;
2(S)-[3-[2(S)-(butoxycarbonyl)-2-hydroxyethyl]-3-isobutyl-ureido]-3-(2-naphthyl)propionic acid;
N-[[1-[(S)-3-[(S)-6-amino-2-methanesulfonamidohexanamido]-2-carboxypropyl]cyclopentyl]carbonyl]-1-tyrosine;
5-[4′-[(3,5-dibutyl-1H-1,2,4-triazol-1-yl)methyl][1,1′-biphenyl]-2-yl]-1H-tetrazole;
N-[1-[hydroxy[1(R)-[Nalpha-(methylsulfonyl)-1-lysylamino]-2-phenylethyl]phosphinylmethyl]cyclopentylcarbonyl]-1-tryptophan dilithium salt;
7-[2-[[1-(ethoxycarbonyl)-3-phenylpropyl]amino]-1-oxopropyl]-,[8S-[7[R*(R*)],8R*]]1,4-dithia-7-azaspiro[4.4]nonane-8-carboxylic acid; and
N-[2-(mercaptomethyl)-1-oxo-3-phenylpropyl]-L-leucine,
or an isomer, a pharmaceutically acceptable salt, ester, or prodrug thereof.

9. The method of claim 1 wherein the cyclooxygenase-2 selective inhibitor and the ACE inhibitor are administered substantially simultaneously.

10. The method of claim 1 wherein the cyclooxygenase-2 selective inhibitor and the ACE inhibitor are administered sequentially.

11. The method of claim 1 wherein the cyclooxygenase-2 selective inhibitor is administered to the subject in an amount of about 0.1 to about 20 mg/kg body weight per day.

12. The method of claim 1 wherein the ACE inhibitor is administered to the subject in an amount of about 5 to about 500 milligrams per day.

13. A method for treating a stroke, the method comprising:
(a) diagnosing a subject in need of treatment for a stroke; and
(b) administering to the subject an ACE inhibitor or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a chromene compound, the chromene compound comprising a benzothiopyran, a dihydroquinoline or a dihydronaphthalene.

14. The method of claim 13 wherein the cyclooxygenase-2 selective inhibitor has a selectivity ratio of COX-1 IC₅₀ to COX-2 IC₅₀ not less than about 50.

15. The method of claim 14 wherein the cyclooxygenase-2 selective inhibitor has a selectivity ratio of COX-1 IC₅₀ to COX-2 IC₅₀ not less than about 100.

16. The method of claim 13 wherein the cyclooxygenase-2 selective inhibitor or an isomer, a pharmaceutically acceptable salt, ester, or prodrug thereof is a compound having the formula: 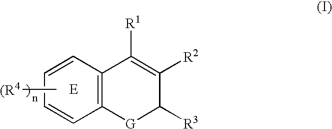
wherein:
n is an integer which is 0, 1, 2, 3 or 4;
G is O, S or NRa;
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or R⁴ together with the carbon atoms to which it is attached and the remainder of ring E forms a naphthyl radical.

17. The method of claim 13 wherein the cyclooxygenase-2 selective inhibitor is (S)-6,8-dichloro-2-(trifluoromethyl)-2H-1-benzopyran-3-carboxylic acid or an isomer, a pharmaceutically acceptable salt, ester, or prodrug thereof.

18. The method of claim 13 wherein the ACE inhibitor is selected from the group consisting of
enalapril;
captopril;
benazepril;
fosinopril;
lisinopril;
moexipril;
perindopril;
quinapril;
ramipril;
trandolapril;
1-[3-(benzoylthio)-2-methyl-1-oxopropyl]-4-(phenylthio)-,[1(R*),2alpha,4alpha]-L-proline;
2-[2-[[1-(ethoxycarbonyl)-3-phenylpropyl]amino]-1-oxopropyl]-,[3S-[2[R*(R*)],3R*]]-2-azabicyclo[2.2.2]octane-3-carboxylic acid;
N-[2(S)-benzyl-3-sulfanylpropionyl]4-(2-thiazolyl)-L-phenylalanine;
2S-[1[R*(R*)],2R*]]-1-[2-[[1-(ethoxycarbonyl)-3-phenylpropyl]amino]-1-oxopropyl]-2,3-dihydro-1H-indole-2-carboxylic acid monosodium salt;
5-(1,1-dimethylethyl)-3-[2-[[1-(ethoxycarbonyl)-3-phenylpropyl]amino]-1-oxopropyl]-2,3-dihydro-,[2S[2R*,3[R*(R*)]]]-1,3,4-thiadiazole-2-carboxylic acid;
N-(N2-acetyl-L-lysyl)-L-2-[[1-[([1-carboxy-2-(4-hydroxyphenyl)ethyl]amino]carbonyl]cyclopentyl]methyl]-b-alanine;
N-[[1-(2-carboxy-4-phenylbutyl)cyclopentyl]carbonyl]-L-tryptophan;
1-(N-((1S)-1-carboxy-3-phenylpropyl)-(S)-alanyl-4-(N-methyl-N-(6-chloro-7-sulfamoyl)-3,4-dihydro-1,2,4-benzothiadiazine-1,1-dioxide-3-yl)methyl)aminoproline;
6-[[1-(ethoxycarbonyl)-3-phenylpropyl]amino]tetrahydro-5-oxo-2-(2-thienyl)-,[2S-[2alpha,6beta(R*)]]-1,4-thiazepine-4(5H)-acetic acid;
1-[2-[[1-(ethoxycarbonyl)-3-phenylpropyl]amino]-1-oxopropyl]octahydro-,[2S-[1[R*(R*)],2alpha,3aalpha,7abeta]]-1H-Indole-2-carboxylic acid;
1-[N-(8-amino-1-carboxyoctyl)-L-alanyl]-,(S)-L-proline;
N-[[1-[[2-(acetylthio)-3-methyl-1-oxobutyl]amino]cyclopentyl]carbonyl]-ethyl ester (S)- L-tryptophan;
1-[(2S)-3-(acetylthio)-2-methyl-1-oxopropyl]-L-prolyl-L-phenylalanine;
3-[[(1S)-1-(ethoxycarbonyl)-3-phenylpropyl]amino]-2,3,4,5-tetrahydro-2-oxo- (3S)-1H-1-benzazepine-1-acetic acid;
[S-(R*,R*)]-2,3,4,5-tetrahydro-3-[(2-mercapto-1-oxo-3-phenylpropyl)amino]-2-oxo-1H-benzazepine-1-acetic acid;
N-[4-(2,3-dihydro-2-benzofuranyl)-1-(ethoxycarbonyl)butyl]-L-alanyl-L-proline;
N-[(S)-1-carboxy-5-(4-[(S)-2-hydroxy-3-isopropylaminoproproxy]-1H-indole-2-carboxamido)pentyl]-(S)-alanyl-(S)-proline;
1-[(2S)-3-mercapto-2-methyl-1-oxopropyl]-L-proline;
1-[6-amino-2-[[hydroxy(4-phenylbutyl)phosphinyl]oxy]-1-oxohexyl]-,(S)-L-proline;
(3S,6S)-4-oxo-3-(sulfanylmethyl)-5-azabicyclo[10.3.1]hexadeca-1(16),12,14-triene-6-carboxylic acid;
2,3,4,5,6,7,8,9-octahydro-2-(mercaptomethyl)-3-oxo-,(2S,5S)-1H-4-benzazacycloundecine-5-carboxylic acid;
10-[(3-phenyl-2-mercapto-1-oxo)propylamino]-9-oxo-8-azatricyclo[4.3.2]-1,3,5-hexahydrododecan-7-carboxylic acid;
N-[1-[2(S)-(acetylsulfanyl)-3-methylbutyramido]cyclopent-1-ylcarbonyl]4-O-methyl-L-tyrosine ethyl ester;
9-[[(1S)-1-(ethoxycarbonyl)-3-phenylpropyl]amino]octahydro-10-oxo-(1S,9S)-6H-pyridazino[1,2-a][1,2]diazepine-1-carboxylic acid;
3-[[1-carboxy-5-(4-piperidinyl)pentyl]amino]3,4,dihydro-4-oxo,[s-(R*,S*)]-1,5-benzothiazepine-5(2H)-acetic acid;
N-(2,3-dihydro-1H-inden-2-yl)-N-[N-[1-(ethoxycarbonyl)-3-phenylpropyl]-L-alanyl]-(S)-glycine;
(2S,3aS,7aS)-1-[N-(3-pyridylcarbonyl)-1-lysyl-gamma-dglutamyl]octahydroindole-2-carboxylic acid;
N-[(1S)-1-(ethoxycarbonyl)-3-phenylpropyl]-L-alanyl-L-proline;
N-[(1S)-1-carboxy-3-phenylpropyl]-L-alanyl-L-proline;
(SYN-[N-[1-[ethoxycarbonyl]-3-phenylpropyl]-L-alanyl]-N-[2-ethoxyethoxy]-glycine;
(3R,6S,9R,9aR)-6-[(2S,3S)-2-acetylthio-3-methylpentylamido]-9-methyl-5-oxooctahydroazepino[2,1-b]thiazole-3-carboxylic acid;
1,2,3,4,6,7,8,12b-octahydro-7-[(2-mercapto-3-methyl-1-oxopentyl)amino]-6-oxo-11-phenyl-[4S-[4a,7a(2R*,3R*),12bb]]-pyrido[2,1-a][2]benzazepine-4-carboxylic acid;
octahydro-6-[((2-mercapto-3-methyl-1-oxopentyl)amino)]-5-oxo-,[(3R-[(3alpha,6alpha(2S*,3S*),9alphabeta)])]-thiazolo[(3,2-a)]azepine-3-carboxylic acid;
(−)-(S)-N-[2-acetylthiomethyl)-3-(3,4-methylenedioxyphenyl)propanoyl]-(S)-alanine benzyl ester;
N2-acetyl-N5-formyl-N5-hydroxy-L-ornithyl-N-[3-[(2S,5R)-5-[3-(formylhydroxyamino)propyl]-3,6-dioxo-2-piperazinyl]propyl]-N-hydroxy-L-serinamide;
4-cyclohexyl-1-[[[2-methyl-1-(1-oxopropoxy)propoxy](4-phenylbutyl)phosphinyl]acetyl]-,[1[S*(R*)],2alpha,4beta]-,sodium salt L-proline;
[4(S)-cyclohexyl-2(R)-1-prolin-1yl]carbonylmethyl-(4-phenylbutyl)phosphinic acid;
2(R)-[3-mercapto-2(S)-methylpropranoyloxy]-3-(methylthio)propanoic acid;
hexahydro-6-[[[2S]-2-mercapto-1-oxo-3-phenylpropyl]amino]-2,2-dimethyl-7-oxo-,[6S]-1H-1-acetic acid;
N-[3-(acetylthio)-2-(1,3-benzodioxol-5-ylmethyl)-1-oxopropyl]-,phenylmethyl ester,(S)-glycine;
2-[[[2-(hydroxyamino)-2-oxoethyl]-N-methylamino]carbonyl]-,(+)-cis-(1S,2R)-cyclohexanecarboxylic acid;
3-[2-[[1-(ethoxycarbonyl)-3-phenylpropyl]amino]-1-oxopropyl]-1-methyl-2-oxo-,monohydrochloride[4S-[3[R*(R*)],4R*]]4-imidazolidinecarboxylic acid;
3-[(5-amino-1-carboxypentyl)amino]-2,3,4,5-tetrahydro-2-oxo-,dihydrobromide,[S-(R*,R*)]-1H-1-benzazepine-1-acetic acid;
N2-[(1S)-1-carboxy-3-phenylpropyl]-L-lysyl-,dihydrate L-proline;
7-[(1-carboxy-3-phenylpropyl)amino]-1,2,3,4,6,7,8,12a-octahydro-6-oxo-,[4S-[4alpha,7alpha,(R*),12bbeta]]-pyrido[2,1-a][2]benzazepine-4-carboxylic acid;
4(S)-4alpha,7alpha(R*),12bbeta]-7-[1-(ethoxycarbonyl)-3-phenylpropylamino]-1,2,3,4,5,6,7,8,12b-octahydro-6-oxopyrido[2,1-a][2]benzazepine-4-carboxylic acid diphenylmethyl ester;
(4S,7S,2bR)-7-[2(S)-(acetylthio)-3-phenylpropionamido]-6-oxo-1,2,3,4,6,7,8,12b-octahydropyrido[2,1-a][2]benzazepine-4-carboxylic acid;
6-oxo-7(S)-[3-phenyl-2(R)-sulfanylpropanamido]-6-oxo-1,2,3,4,6,7,8,12b(R)-octahydropyrido[2,1-a]-2-benzazepine-4(S)-carboxylic acid;
7-[[[2-(carboxymethyl)-2,3-dihydro-1H-inden-2-yl]carbonyl]amino]-1,2,3,4,6,7,8,12b-octahydro-6-oxo-,(4S,7S,12bR)-Pyrido[2,1-a][2]benzazepine-4-carboxylic acid;
N-[(2S,3R)-2-benzoylthiomethyl-1-oxo-3-phenylbutyl]-(S)-alanine;
2-[2-[[1-(ethoxycarbonyl)-3-phenylpropyl]amino]-1-oxopropyl]-1,2,3,4-tetrahydro-6,7-dimethoxy-,[3S-[2[R*(R*)],3R*]]-monohydrochloride-3-isoquinolinecarboxylic acid;
2-[(2S)-2-[[(1S)-1-carboxy-3-phenylpropyl]amino]-1-oxopropyl]-1,2,3,4-tetrahydro-6,7-dimethoxy-,(3S)-3-isoquinolinecarboxylic acid;
1-[3-[[2-[(cyclohexylcarbonyl)amino]-1-oxopropyl]thio]-2-methyl-1-oxopropyl]-,calcium salt (2:1)[R-(R*,S*)]-L-proline;
1-[2-methyl-3-(nitrosothio)-1-oxopropyl]-,(S)-L-proline;
(4S,7S,10aS)-octahydro-4-[(S)-alpha-mercaptohydrocinnamido]-5-oxo-7H-pyrido[2,1-b][1,3]thiazepine-7-carboxylic acid;
1-[(2S)-2-[[(1S)-1-(ethoxycarbonyl)butyl]amino]-1-oxopropyl]octahydro-,(2S,3aS,7aS)-1H-indole-2-carboxylic acid;
(2S,3aS ,7aS)-1-[(S)-N-[(S)-1-carboxybutyl]alanyl]hexahydro-2-indolinecarboxylic acid;
2-[2[(-1-carboxy-3-phenylpropyl)amino-1-oxopropyl]-1,2,3,4-tetrahydro-,[3S-[2[R*(R*)],3R*]]-3-isoquinolinecarboxylic acid;
1-[2-[[1-(ethoxycarbonyl)-3-phenylpropyl]amino]-1-oxopropyl]octahydro-,[2S-[1[R*(R*)],2alpha,3abeta,6abeta]]-cyclopenta[b]pyrrole-2-carboxylic acid;
N-[2(R)-mercapto-1-oxo-3-(4-oxyphenyl)propyl-glycyl]-[4(S)-(3-oxyphenyl)]-proline;
2-(2-hydroxyphenyl)-3-(3-mercapto-1-oxopropyl)-,(2R,4R)-4-thiazolidinecarboxylic acid;
N-acetyl-L-a-aspartyl-L-phenylalanyl-y(PO(OH)—CH2)-L-alanyl-L-alaninamide;
2(S)-[3-[2(S)-carboxy-2-hydroxyethyl]-3-isobutylureido]-3-(2-naphthyl)propionic acid;
2(S)-[3-[2(S)-(butoxycarbonyl)-2-hydroxyethyl]-3-isobutyl-ureido]-3-(2-naphthyl)propionic acid;
N-[[1-[(S)-3-[(S)-6-amino-2-methanesulfonamidohexanamido]-2-carboxypropyl]cyclopentyl]carbonyl]-1-tyrosine;
5-[4′-[(3,5-dibutyl-1H-1,2,4-triazol-1-yl)methyl][1,1′-biphenyl]-2-yl]-1H-tetrazole;
N-[1-[hydroxy[1(R)-[Nalpha-(methylsulfonyl)-1-lysylamino]-2-phenylethyl]phosphinylmethyl]cyclopentylcarbonyl]-1-tryptophan dilithium salt;
7-[2-[[1-(ethoxycarbonyl)-3-phenylpropyl]amino]-1-oxopropyl]-,[8S-[7[R*(R*)],8R*]]1,4-dithia-7-azaspiro[4.4]nonane-8-carboxylic acid; and
N-[2-(mercaptomethyl)-1-oxo-3-phenylpropyl]-L-leucine,
or an isomer, a pharmaceutically acceptable salt, ester, or prodrug thereof.

19. A method for treating a stroke, the method comprising:
(a) diagnosing a subject in need of treatment for a stroke; and
(b) administering to the subject an ACE inhibitor or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a phenyl acetic acid compound.

20. The method of claim 19 wherein the cyclooxygenase-2 selective inhibitor has a selectivity ratio of COX-1 IC₅₀ to COX-2 IC₅₀ not less than about 50.

21. The method of claim 20 wherein the cyclooxygenase-2 selective inhibitor has a selectivity ratio of COX-1 IC50 to COX-2 IC₅₀ not less than about 100.

22. The method of claim 19 wherein the cyclooxygenase-2 selective inhibitor or an isomer, a pharmaceutically acceptable salt, ester, or prodrug thereof is a compound having the formula: 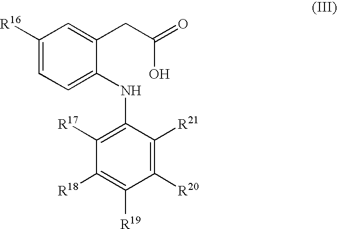
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl; provided, however, that each of R¹⁷, R¹⁸, R²⁰ and R²¹ is not fluoro when R¹⁶ is ethyl and R¹⁹ is H.

23. The method of claim 22 wherein R¹⁶ is ethyl, R¹⁷ and R¹⁹ are chloro, R¹⁸ and R²⁰ are hydrogen, and R²¹ is methyl.

24. The method of claim 19 wherein the ACE inhibitor is selected from the group consisting of:
enalapril;
captopril;
benazepril;
fosinopril;
lisinopril;
moexipril;
perindopril;
quinapril;
ramipril;
trandolapril;
1-[3-(benzoylthio)-2-methyl-1-oxopropyl]-4-(phenylthio)-,[1(R*),2alpha,4alpha]-L-proline;
2-[2-[[1-(ethoxycarbonyl)-3-phenylpropyl]amino]-1-oxopropyl]-,[3S-[2[R*(R*)],3R*]]-2-azabicyclo[2.2.2]octane-3-carboxylic acid;
N-[2(S)-benzyl-3-sulfanylpropionyl]4-(2-thiazolyl)-L-phenylalanine;
2S-[1[R*(R*)],2R*]]-1-[2-[[1-(ethoxycarbonyl)-3-phenylpropyl]amino]-1-oxopropyl]-2,3-dihydro-1H-indole-2-carboxylic acid monosodium salt;
5-(1,1-dimethylethyl)-3-[2-[[1-(ethoxycarbonyl)-3-phenylpropyl]amino]-1-oxopropyl]-2,3-dihydro-,[2S[2R*,3[R*(R*)]]]-1,3,4-thiadiazole-2-carboxylic acid;
N-(N2-acetyl-L-lysyl)-L-2-[[1-[[[1-carboxy-2-(4-hydroxyphenyl)ethyl] amino]carbonyl]cyclopentyl]methyl]-b-alanine;
N-[[1-(2-carboxy-4-phenylbutyl)cyclopentyl]carbonyl]-L-tryptophan;
1-(N-((1S)-1-carboxy-3-phenylpropyl)-(S)-alanyl-4-(N-methyl-N-(6-chloro-7-sulfamoyl)-3,4-dihydro-1,2,4-benzothiadiazine-1,1-dioxide-3-yl)methyl)aminoproline;
6-[[1-(ethoxycarbonyl )-3-phenylpropyl]amino]tetrahydro-5-oxo-2-(2-thienyl)-,[2S-[2alpha,6beta(R*)]]-1,4-thiazepine-4(5H)-acetic acid;
1-[2-[[1-(ethoxycarbonyl)-3-phenylpropyl]amino]-1-oxopropyl]octahydro-,[2S-[1[R*(R*)],2alpha,3aalpha,7abeta]]-1H-Indole-2-carboxylic acid;
1-[N-(8-amino-1-carboxyoctyl)-L-alanyl]-,(S)-L-proline;
N-[[1-[[2-(acetylthio)-3-methyl-1-oxobutyl]amino]cyclopentyl]carbonyl]-ethyl ester(S)-L-tryptophan;
1-[(2S)-3-(acetylthio)-2-methyl-1-oxopropyl]-L-prolyl-L-phenylalanine;
3-[[(1S)-1-(ethoxycarbonyl)-3-phenylpropyl]amino]-2,3,4,5-tetrahydro-2-oxo-(3S)-1H-1-benzazepine-1-acetic acid;
[S-(R*,R*)]-2,3,4,5-tetrahydro-3-[(2-mercapto-1-oxo-3-phenylpropyl)amino]-2-oxo-1H-benzazepine-1-acetic acid;
N-[4-(2,3-dihydro-2-benzofuranyl)-1-(ethoxycarbonyl)butyl]-L-alanyl-L-proline;
N-[(S)-1-carboxy-5-(4-[(S)-2-hydroxy-3-isopropylaminoproproxy]-1H-indole-2-carboxamido)pentyl]-(S)-alanyl-(S)-proline;
1-[(2S)-3-mercapto-2-methyl-1-oxopropyl]-L-proline;
1-[6-amino-2-[[hydroxy(4-phenylbutyl)phosphinyl]oxy]-1-oxohexyl]-,(S)-L-proline;
(3S,6S)4-oxo-3-(sulfanylmethyl)-5-azabicyclo[10.3.1]hexadeca-1(16), 12,14-triene-6-carboxylic acid;
2,3,4,5,6,7,8,9-octahydro-2-(mercaptomethyl)-3-oxo-,(2S,5S)-1H4-benzazacycloundecine-5-carboxylic acid;
10-[(3-phenyl-2-mercapto-1-oxo)propylamino]-9-oxo-8-azatricyclo[4.3.2]-1,3,5-hexahydrododecan-7-carboxylic acid;
N-[1-[2(S)-(acetylsulfanyl)-3-methylbutyramido]cyclopent-1-ylcarbonyl]-4-O-methyl-L-tyrosine ethyl ester;
9-[[(1S)-1-(ethoxycarbonyl)-3-phenylpropyl]amino]octahydro-10-oxo-(1S,9S)-6H-pyridazino[1,2-a][1,2]diazepine-1-carboxylic acid;
3-[[1-carboxy-5-(4-piperidinyl)pentyl]amino]3,4,dihydro-4-oxo,[s-(R*,S*)]-1,5-benzothiazepine-5(2H)-acetic acid;
N-(2,3-dihydro-1H-inden-2-yl)-N-[N-[1-(ethoxycarbonyl)-3-phenylpropyl]-L-alanyl]-(S)-glycine;
(2S ,3aS,7aS)-1-[N-(3-pyridylcarbonyl)-1-lysyl-gamma-dglutamyl]octahydroindole-2-carboxylic acid;
N-[(1S)-1-(ethoxycarbonyl)-3-phenylpropyl]-L-alanyl-L-proline;
N-[(1S)-1-carboxy-3-phenylpropyl]-L-alanyl- L-proline;
(S)-N-[N-[1-[ethoxycarbonyl]-3-phenylpropyl]-L-alanyl]-N-[2-ethoxyethoxy]-glycine;
(3R,6S,9R,9aR)-6-[(2S,3S)-2-acetylthio-3-methylpentylamido]-9-methyl-5-oxooctahydroazepino[2,1-b]thiazole-3-carboxylic acid;
1,2,3,4,6,7,8,12b-octahydro-7-[(2-mercapto-3-methyl-1-oxopentyl)amino]-6-oxo-11-phenyl-[4S-[4a,7a(2R*,3R*), 12bb]]-pyrido[2,1-a][2]benzazepine-4-carboxylic acid; octahydro-6-[((2-mercapto-3-methyl-1-oxopentyl)amino)]-5-oxo-,[(3R-[(3alpha,6alpha(2S*,3S*),9alphabeta)])]-thiazolo[(3,2-a)]azepine-3-carboxylic acid;
(−)-(S)-N-[2-acetylthiomethyl)-3-(3,4-methylenedioxyphenyl)propanoyl]-(S)-alanine benzyl ester;
N2-acetyl-N5-formyl-N5-hydroxy-L-ornithyl-N-[3-[(2S,5R)-5-[3-(formylhydroxyamino)propyl]-3,6-dioxo-2-piperazinyl]propyl]-N-hydroxy-L-serinamide;
4-cyclohexyl-1-[[[2-methyl-1-(1-oxopropoxy)propoxy](4-phenylbutyl)phosphinyl]acetyl]-[1[S*(R*)],2alpha,4beta]-,sodium salt L-proline;
[4(S)-cyclohexyl-2(R)-I-prolin-1yl]carbonylmethyl-(4-phenylbutyl)phosphinic acid;
2(R)-[3-mercapto-2(S)-methylpropranoyloxy]-3-(methylthio)propanoic acid;
hexahydro-6-[[[2S]-2-mercapto-1-oxo-3-phenylpropyl]amino]-2,2-dimethyl-7-oxo-,[6S]-1 H-1-acetic acid;
N-[3-(acetylthio)-2-(1,3-benzodioxol-5-ylmethyl)-1-oxopropyl]-,phenylmethyl ester,(S)-glycine;
2-[[[2-(hydroxyamino)-2-oxoethyl]-N-methylamino]carbonyl]-,(+)-cis-(1S,2R)-cyclohexanecarboxylic acid;
3-[2-[[1-(ethoxycarbonyl)-3-phenylpropyl]amino]-1-oxopropyl]-1-methyl-2-oxo-,monohydrochloride[4S-[3[R*(R*)],4R*]]-4-imidazolid inecarboxylic acid;
3-[(5-amino-1-carboxypentyl)amino]-2,3,4,5-tetrahydro-2-oxo-,dihydrobromide,[S-(R*,R*)]-1H-1-benzazepine-1-acetic acid;
N2-[(1S)-1-carboxy-3-phenylpropyl]-L-lysyl-,dihydrate L-proline;
7-[(1-carboxy-3-phenylpropyl)amino]-1,2,3,4,6,7,8,12a-octahydro-6-oxo-,[4S-[4alpha,7alpha,(R*),12bbeta]]-pyrido[2,1-a][2]benzazepine4-carboxylic acid;
4(S)-4alpha,7alpha(R*),12bbeta]-7-[1-(ethoxycarbonyl)-3-phenylpropylamino]-1,2,3,4,5,6,7,8,12b-octahydro-6-oxopyrido[2,1-a][2]benzazepine-4-carboxylic acid diphenylmethyl ester;
(4S,7S,12bR)-7-[2(S)-(acetylthio)-3-phenylpropionamido]-6-oxo-1,2,3,4,6,7,8,12b-octahydropyrido[2,1-a][2]benzazepine-4-carboxylic acid;
6-oxo-7(S)-[3-phenyl-2(R)-sulfanylpropanamido]-6-oxo-1,2,3,4,6,7,8,12b(R)-octahydropyrido[2,1-a]-2-benzazepine-4(S)-carboxylic acid;
7-[[[2-(carboxymethyl)-2,3-dihydro-1H-inden-2-yl]carbonyl]amino]-1,2,3,4,6,7,8,12b-octahydro-6-oxo-,(4S,7S,12bR)-Pyrido[2,1-a][2]benzazepine-4-carboxylic acid;
N-[(2S,3R)-2-benzoylthiomethyl-1-oxo-3-phenylbutyl]-(S)-alanine;
2-[2-[[1-(ethoxycarbonyl)-3-phenylpropyl]amino]-1-oxopropyl]-1,2,3,4-tetrahydro-6,7-dimethoxy-,[3S-[2[R*(R*)],3R*]]-monohydrochloride-3-isoquinolinecarboxylic acid;
2-[(2S)-2-[[(1S)-1-carboxy-3-phenylpropyl]amino]-1-oxopropyl]-1,2,3,4-tetrahydro-6,7-dimethoxy-,(3S)-3-isoquinolinecarboxylic acid;
1-[3-[[2-[(cyclohexylcarbonyl)amino]-1-oxopropyl]thio]-2-methyl-1-oxopropyl]-,calcium salt (2:1) [R-(R*,S*)]-L-proline;
1-[2-methyl-3-(nitrosothio)-1-oxopropyl]-,(S)-L-proline;
(4S,7S,10aS)-octahydro-4-[(S)-alpha-mercaptohydrocinnamido]-5-oxo-7H-pyrido[2,1-b][1,3]thiazepine-7-carboxylic acid;
1-[(2S)-2-[[(1S)-1-(ethoxycarbonyl)butyl]amino]-1-oxopropyl]octahydro-, (2S,3aS,7aS)-1 H-indole-2-carboxylic acid;
(2S,3aS,7aS)-1-[(S)-N-[(S)-1-carboxybutyl]alanyl]hexahydro-2-indolinecarboxylic acid;
2-[2[(-1-carboxy-3-phenylpropyl)amino-1-oxopropyl]-1,2,3,4-tetrahydro-,[3S-[2[R*(R*)],3R*]]-3-isoquinolinecarboxylic acid;
1-[2-[[1-(ethoxycarbonyl)-3-phenylpropyl]amino]-1-oxopropyl]octahydro-,[2S-[1[R*(R*)],2alpha,3abeta,6abeta]]-cyclopenta[b]pyrrole-2-carboxylic acid;
N-[2(R)-mercapto-1-oxo-3-(4-oxyphenyl)propyl-glycyl]-[4(S)-(3-oxyphenyl)]-proline;
2-(2-hydroxyphenyl)-3-(3-mercapto-1-oxopropyl)-,(2R,4R)-4-thiazolidinecarboxylic acid;
N-acetyl-L-a-aspartyl-L-phenylalanyl-y(PO(OH)—CH2)-L-alanyl-L-alaninamide;
2(S)-[3-[2(S)-carboxy-2-hydroxyethyl]-3-isobutylureido]-3-(2-naphthyl)propionic acid;
2(S)-[3-[2(S)-(butoxycarbonyl)-2-hydroxyethyl]-3-isobutyl-ureido]-3-(2-naphthyl)propionic acid;
N-[[1-[(S)-3-[(S)-6-amino-2-methanesulfonamidohexanamido]-2-carboxypropyl]cyclopentyl]carbonyl]-1-tyrosine;
5-[4′-[(3,5-dibutyl-1H-1,2,4-triazol-1-yl)methyl][1,1′-biphenyl]-2-yl]-1H-tetrazole;
N-[1-[hydroxy[1(R)-[Nalpha-(methylsulfonyl)-1-lysylamino]-2-phenylethyl]phosphinylmethyl]cyclopentylcarbonyl]-1-tryptophan dilithium salt;
7-[2-[[1-(ethoxycarbonyl)-3-phenylpropyl]amino]-1-oxopropyl]-,[8S-[7[R*(R*)],8R*]]1,4-dithia-7-azaspiro[4.4]nonane-8-carboxylic acid; and
N-[2-(mercaptomethyl)-1-oxo-3-phenylpropyl]-L-leucine, or an isomer, a pharmaceutically acceptable salt, ester, or prodrug thereof.

25. A method for treating a stroke, the method comprising:
(a) diagnosing a subject in need of treatment for a stroke; and
(b) administering to the subject a chromene or phenyl acetic acid cyclooxygenase-2 selective inhibitor selected from the group consisting of (S)-6,8-dichloro-2-(trifluoromethyl)-2H-1-benzopyran-3-carboxylic acid, 6-nitro-2-triflouromethyl-2H-1-benzopyran-3-carboxylic acid, 6-chloro-8-methyl-2-triflouromethyl-2H-1-benzopyran-3-carboxylic acid, 6-chloro-2-trifluoromethyl-2H-1-benzopyran-3-carboxylic acid, 6-chloro-7-methyl-2-trifluoromethyl-2H-1-benzopyran-3-carboxylic acid, 8-(1-methylethyl)-2-trifluoromethyl-2H-1-benzopyran-3-carboxylic acid, 6,8-bis(dimethylethyl)-2-trifluoromethyl-2H-1-benzopyran-3-carboxylic acid, 7-phenyl-2-trifluoromethyl-2H-1-benzopyran-3-carboxylic acid, 8-bromo-5-fluoro-2-trifluoromethyl-2H-1-benzopyran-3-carboxylic acid, N-(4-Nitro-2-phenoxy-phenyl)-methanesulfonamide, N-[6-(2,4-difluoro-phenoxy)-1-oxo-indan-5-yl]-methanesulfonamide, N-[5-(4-fluoro-phenylsulfanyl)-thiophen-2-yl]-methanesulfonamide, (5Z)-2-amino-5-[[3,5-bis(1,1-dimethylethyl)-4-hydroxyphenyl]methylene]-4(5H)-thiazolone, N-[3-(formylamino)-4-oxo-6-phenoxy-4H-1-benzopyran-7-yl]-methanesulfonamide, and 6-dioxo-9H-purin-8-yl-cinnamic acid, or an isomer, a pharmaceutically acceptable salt, ester, or prodrug thereof; and
an ACE inhibitor selected from the group consisting of enalapril, captopril, benazepril, fosinopril, lisinopril, moexipril, perindopril, quinapril, ramipril, and trandolapril, or an isomer, a pharmaceutically acceptable salt, ester, or prodrug thereof.

26. The method of claim 25 wherein the cyclooxygenase-2 selective inhibitor and the ACE inhibitor are combined and administered in the same dose.

27. The method of claim 25 wherein the cyclooxygenase-2 selective inhibitor and ACE inhibitor are administered in separate doses.
